(19) United States
(12) Patent Application Publication
(10) Pub. No.: US 2025/0064297 A1
(43) Pub. Date: Feb. 27, 2025

(54) CONTINUUM ROBOT CONTROL DEVICE, CONTINUUM ROBOT CONTROL METHOD, AND PROGRAM

(71) Applicant: CANON KABUSHIKI KAISHA, Tokyo (JP)

(72) Inventor: Hidekazu Kose, Tokyo (JP)

(21) Appl. No.: 18/943,108

(22) Filed: Nov. 11, 2024

Related U.S. Application Data
(63) Continuation of application No. 17/040,923, filed on Sep. 23, 2020, now Pat. No. 12,156,634.

(30) Foreign Application Priority Data
Mar. 23, 2018 (JP) 2018-056793
Jan. 31, 2019 (JP) 2019-016022

(51) Int. Cl. A61B 1/00; A61B 1/005; B25J 9/10; B25J 18/06

(52) U.S. Cl. CPC A61B 1/00006; A61B 1/0057; B25J 9/104; B25J 18/06

(57) ABSTRACT

A continuum robot control device, configured to control operations of a continuum robot having a bendable portion that is bent by driving at least part of a plurality of wires, includes a kinematics computing unit that computes a driving amount l_{k1b} of the at least part of the plurality of wires, based on a target bending angle that is a target value for a bending angle of the bendable portion, a compensation amount computing unit that computes a compensation amount for compensation of the driving amount l_{k1b}, based on the target bending angle, and a displacement of one of the plurality of wires at the target bending angle, and an adding unit and position control unit that set a driving control amount of performing driving control of the at least part of the plurality of wires, based on the driving amount and the compensation amount obtained by computation.

### CROSS REFERENCE OF RELATED APPLICATIONS

This application is a continuation of U.S. patent application Ser. No. 17/040,923, filed on Sep. 23, 2020, which is the National Phase application of PCT Application No. PCT/JP2019/010331, filed on Mar. 13, 2019, which claims the benefit of Japanese Patent Application No. 2018-056793, filed on Mar. 23, 2018, and Japanese Patent Application No. 2019-016022, filed on Jan. 31, 2019, all of which are hereby incorporated by reference herein in their entirety.

### TECHNICAL FIELD

The present invention relates to a continuum robot control device and a continuum robot control method that controls operations of a continuum robot having bendable portions that can bend by wires being driven, and a program that causes a computer to function as the continuum robot control device.

### BACKGROUND ART

In recent years, there is increased interest in minimally invasive medical care to reduce the load on the patient and improve the quality of life (QOL) following treatment or tests. A representative example of minimally invasive medical care is surgery/testing using endoscopes. For example, laparoscopic surgery leaves a smaller surgical wound as compared with conventional abdominal surgery, so not only can the necessary hospitalization period following surgery be shortened, but there is also an advantage of better aesthetics as well.

Flexible endoscopes are known as endoscopes used in minimally invasive medical care. These endoscopes have the portion that is inserted configured of a bendable member, so deep portions of the body can be reached without compressing tissue even in bended organs such as the esophagus, large intestine, and lungs. It is further anticipated that using actuators and the like to drive the insertion portion, and control the attitude thereof to follow paths in the body will be able to further reduce the load on the patient. Accordingly, research and development of continuum robot mechanisms usable as a flexible endoscope and control methods thereof are being actively pursued.

Such continuum robots may employ an arrangement where actuators are disposed on a base by using driving force transmission mechanism such as wires or the like, for example, thereby reducing the diameter of the bendable portion. For example, PTL 1 describes a method regarding a continuum robot using wires for a driving force transmission mechanism, where controlling the driving amount of the wires matches the attitude of a bendable portion to a target posture. To do so, the driving amount of the wires is computed using kinematics in PTL 1, assuming the wires to be rigid in the longitudinal direction.

In order to realize a continuum robot that can reach narrow spaces in deep parts of the body, such as the lungs, the wires serving as the driving transmission mechanism preferably are long and thin. However, such wires deform greatly under tensile force occurring at the time of pushing and pulling. Accordingly, there has been a problem that performing control input of driving amount of the wires computed using kinematics, such as the technology described in the specification of PTL 1, results in error occurring between the target posture of the bendable portion and the actual curvature, and the risk of contact between the wall of the body cavity and the bendable portion increases.

### CITATION LIST

### Patent Literature

PTL 1: U.S. Pat. No. 9,333,650

### SUMMARY OF INVENTION

The present invention provides an arrangement that realizes reduced risk of contact between the wall of the body cavity (path within the body, etc.) and the bendable portion.

### Solution to Problem

A continuum robot control device according to the present invention is a continuum robot control device configured to control operations of a continuum robot having a bendable portion that is bent by driving at least part of a plurality of wires. The continuum robot control device includes a first computing device configured to compute a driving amount of the at least part of the plurality of wires, based on a target bending angle that is a target value for a bending angle of the bendable portion, a second computing device configured to compute a compensation amount for compensation of the driving amount, based on the target bending angle, and a displacement of one of the plurality of wires at the target bending angle, and a setting device configured to set a driving control amount of performing driving control of the at least part of the plurality of wires, based on the driving amount calculated at the first computing device and the compensation amount calculated at the second computing device.

Further features of the present invention will become apparent from the following description of exemplary embodiments with reference to the attached drawings.

### BRIEF DESCRIPTION OF DRAWINGS

FIG. 1 is a diagram illustrating an example of the external configuration of a continuum robot used in a first embodiment of the present invention.

FIG. 2 is a diagram illustrating an example of a schematic configuration of a bendable portion illustrated in FIG. 1, according to the first embodiment of the present invention.

FIG. 3 is a diagram illustrating an example of kinematic model of the bendable portion illustrated in FIG. 2, according to the first embodiment of the present invention.

FIG. 4 is a diagram illustrating an example of the functional configuration of a continuum robot control system according to the first embodiment of the present invention.

FIG. 5 is a characteristics diagram illustrating the relation between target bending angle and angle error according to the first embodiment of the present invention.

FIG. 6 is a characteristics diagram illustrating the relation between wire length and angle error according to the first embodiment of the present invention.

FIG. 7 is a diagram illustrating an example of a schematic configuration of a continuum robot used in a second embodiment of the present invention.

FIG. 8 is a diagram illustrating an example of a kinematics model at a first bendable portion and a second bendable portion illustrated in FIG. 7, according to the second embodiment of the present invention.

FIG. 9 is a diagram illustrating an example of the functional configuration of a continuum robot control system according to the second embodiment of the present invention.

FIG. 10 is a diagram illustrating an example of the functional configuration of a compensation amount computing unit illustrated in FIG. 9, according to the second embodiment of the present invention.

FIG. 11 is a diagram illustrating an example of the functional configuration of a displacement updating unit illustrated in FIG. 10, according to the second embodiment of the present invention.

FIG. 12 is a characteristics diagram illustrating the relation between iteration count, which is a count of iterative calculations performed, and displacement, according to the second embodiment of the present invention.

FIG. 13A is a characteristics diagram illustrating the relation between target bending angle and angle error at the first bendable portion and second bendable portion according to the second embodiment of the present invention.

FIG. 13B is a characteristics diagram illustrating the relation between target bending angle and angle error at the first bendable portion and second bendable portion according to the second embodiment of the present invention.

FIG. 14 is a diagram illustrating an example of a schematic configuration of a continuum robot used in a third embodiment of the present invention.

FIG. 15 is a diagram illustrating an example of kinematic model of the bendable portion illustrated in FIG. 14, according to the third embodiment of the present invention.

FIG. 16 is a diagram illustrating an example of the functional configuration of a continuum robot control system according to the third embodiment of the present invention.

FIG. 17 is a diagram illustrating an example of the functional configuration of a compensation amount computing unit illustrated in FIG. 16, according to the third embodiment of the present invention.

FIG. 18 is a diagram illustrating an example of the functional configuration of a displacement updating unit illustrated in FIG. 17, according to the third embodiment of the present invention.

FIG. 19 is a characteristics diagram illustrating the relation between target bending angle and bending angle error according to the third embodiment of the present invention.

FIG. 20 is a characteristics diagram illustrating the relation between target rotation angle and bending angle error according to the third embodiment of the present invention.

FIG. 21 is a diagram illustrating an example of a schematic configuration of a continuum robot used in a fourth embodiment of the present invention.

FIG. 22 is a diagram illustrating an example of the functional configuration of a continuum robot control system according to the fourth embodiment of the present invention.

FIG. 23 is a diagram illustrating an example of the external configuration of a continuum robot control system according to a fifth embodiment of the present invention.

FIG. 24 is a diagram illustrating an example of kinematic model of the bendable portion illustrated in FIG. 23, according to the fifth embodiment of the present invention.

FIG. 25 is a diagram illustrating an example of the functional configuration of a continuum robot control system according to the fifth embodiment of the present invention.

FIG. 26A is a diagram illustrating an example of the schematic configuration of a continuum robot control system used in a sixth embodiment of the present invention.

FIG. 26B is a diagram illustrating an example of the schematic configuration of a continuum robot control system used in the sixth embodiment of the present invention.

FIG. 27 is a diagram illustrating an example of the functional configuration of a continuum robot control system according to the sixth embodiment of the present invention.

FIG. 28 is a diagram illustrating an example of the functional configuration of a compensation amount computing unit illustrated in FIG. 27, according to the sixth embodiment of the present invention.

FIG. 29 is a diagram illustrating an example of the functional configuration of a displacement updating unit illustrated in FIG. 28, according to the sixth embodiment of the present invention.

FIG. 30 is a characteristics diagram illustrating the relation between target rotation angle and bending angle error according to the sixth embodiment of the present invention.

FIG. 31 is a diagram illustrating an example of the functional configuration of a continuum robot control system according to a seventh embodiment of the present invention.

FIG. 32 is a characteristics diagram illustrating the relation between target rotation angle and drive control amount, according to the seventh embodiment of the present invention.

FIG. 33 is a characteristics diagram illustrating the relation between target bending angle and bending angle error according to the seventh embodiment of the present invention.

### DESCRIPTION OF EMBODIMENTS

Embodiments of the present invention will be described below with reference to the drawings. Specifically, the embodiments of the present invention which will be described below illustrate examples of a control system of a continuum robot (also referred to as a continuum manipulator) having been applied to a flexible endoscope. Note that the flexible endoscope applied as an example of the continuum robot control system according to the embodiments of the present invention is not restricted to the medical field, and is applicable to other fields as long as for observing within a path into which a bendable portion is inserted and removed from (e.g., an industrial endoscope for observing within piping or the like).

### First Embodiment

First, a first embodiment of the present invention will be described.

### (1-1. Modelling)

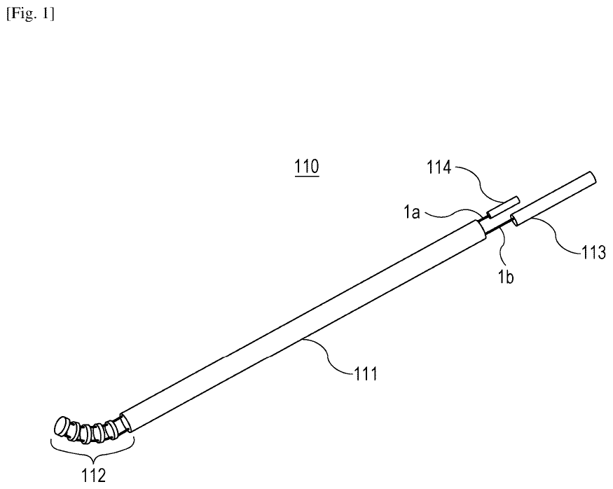

FIG. 1 is a diagram illustrating an example of the external configuration of a continuum robot 110 used in the first embodiment of the present invention. The continuum robot 110 illustrated in FIG. 1 has a wire 1a, a wire 1b, an elongated portion 111, a bendable portion 112, an actuator 113, and a fixing portion 114.

The elongated portion 111 is a component that passively bends under external force. The bendable portion 112 is a component that can be bent by driving part of the multiple wires 1a and 1b, i.e., the wire 1b (predetermined wire). The actuator 113 is a component that drives the wire 1b. The fixing portion 114 is a component that fixes the wire 1a.

In the following description the end portion of the components of the continuum robot 110 that is at the far side from a face of a base (e.g., from the face at the end of the elongated portion 111 on the side of the actuator 113 and fixing portion 114) will be referred to as “distal end”, and the end portion at the side closer to the face of the aforementioned base will be referred to as “proximal end”.

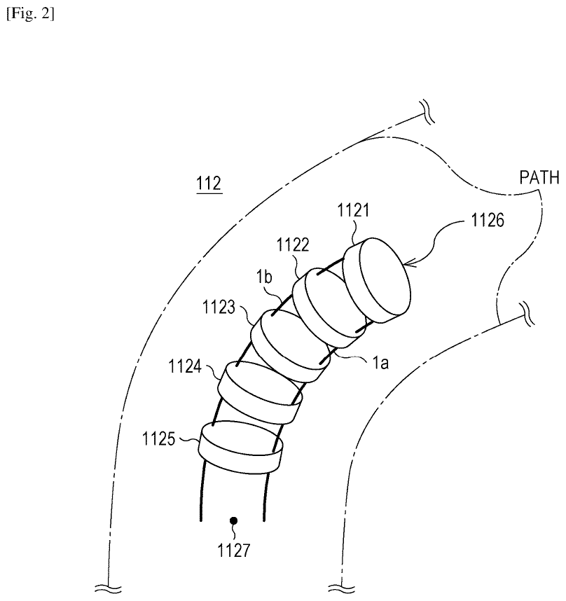

FIG. 2 is a diagram illustrating an example of a schematic configuration of the bendable portion 112 illustrated in FIG. 1, according to the first embodiment of the present invention. FIG. 2 also illustrates an example of a path into which the bendable portion 112 is inserted and removed from.

The bendable portion 112 has the multiple wires 1a and 1b extending through the face at the proximal end of the bendable portion 112, a first wire guide 1121 to which the multiple wires 1a and 1b are fixed at different positions, that guides the multiple wires 1a and 1b, second wire guides 1122 through 1125 that are disposed between the face at the proximal end described above and the first wire guide 1121, and that guide the multiple wires 1a and 1b. FIG. 2 also illustrates a distal end 1126 of the bendable portion 112, and a center position 1127 of the proximal end face of the bendable portion 112 described above. Specifically, the wire 1a and wire 1b are introduced into holes provided to the wire guides 1121 through 1125 and the elongated portion 111, with one end thereof being fixed to the first wire guide 1121, the other end of the wire 1a being connected to the fixing portion 114, and the other end of the wire 1b being connected to the actuator 113. When the actuator 113 illustrated in FIG. 1 is driven in the longitudinal direction of the bendable portion 112, the wire 1b is pushed and pulled, and the bendable portion 112 bends.

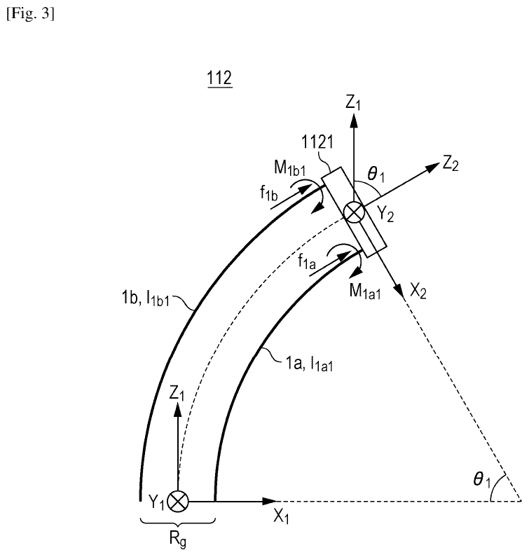

FIG. 3 is a diagram illustrating an example of a kinematic model in the bendable portion 112 illustrated in FIG. 2, according to the first embodiment of the present invention. In FIG. 3, the center position 1127 of the plane of the bendable portion 112 at the proximal end, illustrated in FIG. 2, is the origin. The longitudinal direction is represented by a Z₁ axis, the circumferential direction by an X₁ axis, and the direction from the plane of the drawing toward the far side by a Y₁ axis. In the same way, the center position of the plane of the bendable portion 112 at the distal end 1126, illustrated in FIG. 2, is the origin, the longitudinal direction is represented by a Z₂ axis, the circumferential direction by an X₂ axis, and the direction from the plane of the drawing toward the far side by a Y₂ axis. Further, the angle between the Z₁ axis and Z₂ axis is a bending angle θ₁.

Description will be made below using symbols defined as follows. θ₁ represents the absolute bending angle at the distal end of the bendable portion, l₁ₐ₁ and l_{1b1} represent the length of the wire 1a and the length of the wire 1b of the bendable portion, l_{p1b} represents the drive control amount of the wire 1b of the bendable portion, and 1_{e1b} represents the compensation amount of the wire 1b of the bendable portion. Δl₁ₐ and Δl_{1b} represent the displacement of the wire 1a and the displacement of the wire 1b of the bendable portion, f₁ₐ and f_{1b} represent the force acting on the distal end from the wire 1a and the force acting on the distal end from the wire 1b of the bendable portion, M₁ₐ₁ and M_{1b1} represent the bending moment acting on the distal end from the wire 1a and the bending moment acting on the distal end from the wire 1b of the bendable portion, Rg represents the diameter of a circle of which the radius is the distance from the center of the bendable portion to the wire 1a and wire 1b, L₁₀ is the total length of the wire 1a and wire 1b of the bendable portion, and l₁₀ represents the wire length of the bendable portion when the bending angle is 0 degrees. E represents the Young's modulus of the wires, A represents the cross-sectional area of the wires, and I represents the moment of inertia of area of the wires. Note that for the drive control amount l_{p1b}, the displacements Δl₁ₐ and Δl_{1b}, and forces f₁ₐ and f_{1b}, the distal end direction is the positive direction. The clockwise direction is the positive direction for moments M₁ₐ₁ and M_{1b1}.

The same is true for the following embodiments as well. A kinematic model and elastic wire driving model will be studied in the present embodiment under the following assumptions. Assumption 1 is that at the bendable portion, the wires deform uniformly over the curvature. Assumption 2 is that torsional deformation of the wires is not to be taken into consideration. Assumption 3 is that friction occurring between the wires and wire guides, and between he wires and the elongated portion, is not to be taken into consideration. Assumption 4 is that force and moment acting between the wire guides and wires is not to be taken into consideration. Assumption 5 is that of the forces acting between the wires and the distal end, only the component in the longitudinal direction of the wires is to be taken into consideration, and the component in the radial direction is not to be taken into consideration. Assumption 6 is that the displacement of the wires is proportionate to the tensile force acting on the wires. Assumption 7 is that the bending moment of the wires is proportionate to the deflection angle. Assumption 8 is that tensile rigidity and bending rigidity are equal among the wires. Assumption 9 is that Young's modulus, crossectional area, and moment of inertia of area are equal among the wires.

First, a relational expression that represents a kinematic model will be derived, assuming that the wire 1a and wire 1b are rigid in the longitudinal direction. With the distance between the wire 1a and 1b represented by R_{g} as illustrated in FIG. 3, the bending angle θ₁ can be expressed by the following Expression (1), using the length l₁ₐ₁ of the wire 1a and length l_{1b1} of the wire 1b at the bendable portion 112, based on the above Assumption 1 and Assumption 2.

$${R}_{g} {θ}_{1} = {1}_{1 b 1 -} {1}_{1 a 1} \qquad ( 1 )$$

Now, the right side of Expression (1) is equal to the driving amount of the wire 1b, so by computing using the kinematic model, the driving amount l_{k1b} of the wire 1b can

be expressed by the following Expression (2).

$${l}_{k 1 b} = {R}_{g} {θ}_{1} \qquad ( 2 )$$

Next, a relational expression that represents an elastic wire driving model will be derived, taking into consideration displacement of the wires. The wire length l₁ₐ₁ and the wire length l_{1b1} can be respectively expressed by the following Expressions (3) and (4), where l₁₀ represents the wire length of the bendable portion 112 when the bending angle θ₁ is 0 degrees, Δl₁ₐ represents the displacement of the wire 1a, Δl_{1b} represents the displacement of the wire 1b, and l_{p1b} represents the drive control amount of the wire 1b that is calculated using an elastic wire driving model.

l₁ₐ₁=l₁₀+Δl₁ₐ (3)

$${l}_{1 b 1} = {l}_{10} + Δ {l}_{1 b} + {l}_{p 1 b} \qquad ( 4 )$$

By substituting Expressions (2) through (4) in to Expression (1), the drive control amount l_{p1b} of the wire 1b can be expressed as in the following Expression (5).

$${l}_{p 1 b} = {l}_{k 1 b} + Δ {l}_{1 a} - Δ {l}_{1 b} \qquad ( 5 )$$

Thus, it can be seen from Expression (5) that the drive control amount l_{p1b} of the wire 1b is the sum of the differential of displacement of wires 1a and 1b and the amount of drive l_{k1b}. Accordingly, error in the driving amount due to displacement of the wires can be compensated according to the following Expression (6) as compensation amount l_{e1b}.

$${l}_{e 1 b} = Δ {l}_{1 a} - Δ {l}_{1 b} \qquad ( 6 )$$

Next, the displacement Δl₁ₐ of the wire 1a and the displacement Δl_{1b} of the wire 1b are derived from an expression of balance of the force and moment acting on the distal end 1126 of the bendable portion 112 and the wires. Longitudinal-direction force f₁ₐ and f_{1b} from the wire 1a and wire 1b, and bending moments M₁ₐ₁ and M_{1b1} on the Y₁ axis, act on the distal end 1126 of the bendable portion 112, based on the above Assumptions 3, 4, and 5. Accordingly, the balance of force and moment at the distal end 1126 of the bendable portion 112 can be expressed by the following Expressions (7) and (8).

$${f}_{1 a} + {f}_{1 b} = 0 \qquad ( 7 )$$

$${M}_{1 a 1} + {M}_{1 b 1} - {R}_{g} {f}_{1 a} = 0 \qquad ( 8 )$$

Also, forces f₁ₐ and f_{1b}, and moments M₁ₐ₁ and M_{1b1} can be expressed by the following Expressions (9) and (10), from the above Assumptions 6, 7, and 8, using the displacements Δl₁ₐ and Δl_{1b} and the bending angle θ₁.

$$[ Math . 1 ]$$

$${f}_{1 a} = - {k}_{e} Δ {t}_{1 a} , {f}_{1 b} = - {k}_{e} Δ {l}_{1 b} \qquad ( 9 )$$

$${M}_{1 a 1} = \frac{{k}_{m}}{{l}_{1 a 1}} {θ}_{1} , {M}_{1 b 1} = - \frac{{k}_{m}}{{l}_{1 b 1}} {θ}_{1} \qquad ( 10 )$$

In Expressions (9) and (10), the constants kₑ and kₘ respectively represent the tensile rigidity and bending rigidity of the wires. The constants kₑ and kₘ can be expressed by the following Expressions (11) and (12) using the total length of the wire (wire length) L₀, cross-sectional area A of the wires, moment of inertia of area I, and Young's modulus E, based on the above Assumption 9.

$$\begin{matrix}[ Math \text{.2} ] & \\ {k}_{e} = \frac{AE}{{L}_{0}} & ( 11 )\end{matrix}$$

$${k}_{m} = EI \qquad ( 12 )$$

Also, the relation in the following Expression (13) can be established between the displacement Δl_{1b} of the wire 1b and the displacement Δl₁ₐ of the wire 1a, according to Expressions (7) and (9) in the present embodiment.

Δl_{1b}=−Δl₁ₐ (13)

Accordingly, the compensation amount l_{e1b} of the wire 1b can be expressed by the following Expression (14), from Expressions (6) and (13).

l_{e1b}=2Δl₁ₐ (14)

Substituting Expressions (9) and (10) into Expression (8), and further eliminating l₁ₐ₁, l_{1b1}, Δl_{1b}, and l_{p1b} using Expressions (3), (4), (13), and (14), yields the polynomial for the displacement Δl₁ₐ of the wire 1a, shown in the following Expression (15).

$$\begin{matrix}[ Math \text{.3} ] & \\ \frac{{A}_{3} ( {θ}_{1} ) Δ {l}_{1 a}^{3} + {A}_{2} ( {θ}_{1} ) Δ {l}_{1 a}^{2} + {A}_{1} ( {θ}_{1} ) Δ {l}_{1 a} + {A}_{0} ( {θ}_{1} )}{Δ {l}_{1 a}^{2} + {B}_{1} ( {θ}_{1} ) Δ {l}_{1 a} + {B}_{0} ( {θ}_{1} )} = 0 & ( 15 )\end{matrix}$$

A₃ through A₀ and B₁ through B₀ in Expression (15) are coefficients for the displacement Δl₁ₐ of the wire 1a where the bending angle θ₁ is a variable, and are each expressed in the following Expressions (16) and

$$\begin{matrix}[ Math \text{.4} ] & \\ {A}_{3} ( {θ}_{1} ) = {R}_{g} {k}_{3} , & ( 16 )\end{matrix}$$

$${A}_{2} ( {θ}_{1} ) = {R}_{g}^{2} {k}_{e} {θ}_{1} + 2 {R}_{g} {k}_{3} {l}_{10} ,$$

$${A}_{1} ( {θ}_{1} ) = \{ ( {R}_{g}^{2} {k}_{e} {l}_{10} - 2 {k}_{m} ) {θ}_{1} + {R}_{g} {k}_{e} {l}_{10}^{2} \} ,$$

$${A}_{0} ( {θ}_{1} ) = {R}_{g} {k}_{m} {θ}_{1}^{2} - 2 {k}_{m} {l}_{10} {θ}_{1}$$

$${B}_{1} ( {θ}_{1} ) = {R}_{g} {θ}_{1} + 2 {l}_{10} , \qquad ( 17 )$$

$${B}_{0} ( {θ}_{1} ) = {R}_{g} {l}_{10}^{2} {θ}_{1} + {l}_{10}^{2}$$

The numerator in Expression (15) is a cubic polynomial regarding the displacement Δl₁ₐ of the wire 1a, so solving this yields three different solutions. In the present embodiment, the solution out of these three solutions that has the smallest absolute value is the displacement Δl₁ₐ of the wire 1a.

### (1-2. Control System)

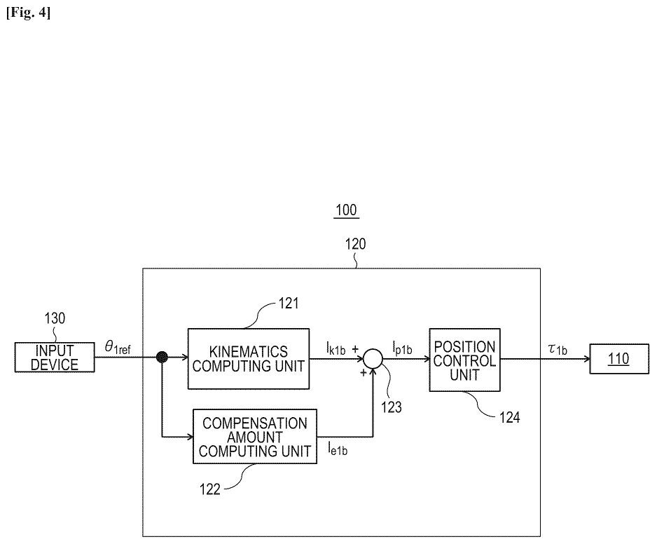

FIG. 4 is a diagram illustrating the functional configuration of a continuum robot control system 100 according to the first embodiment of the present invention. The continuum robot control system 100 illustrated in FIG. 4 is configured including a continuum robot 110 illustrated in FIG. 1, a continuum robot control device 120, and an input device 130.

The continuum robot control device 120 sets an drive control amount l_{p1b} for driving control of the wire 1b, based on a target bending angle θ_{1ref} that is a target value for the bending angle of the bendable portion 112, input from the input device 130, and outputs a drive command τ_{1b} based on the drive control amount lₚ₁ₘ to the continuum robot 110 (or more specifically, to the actuator 113).

The continuum robot control device 120 illustrated in FIG. 4 is configured including a kinematics computing unit 121, a compensation amount computing unit 122, an adding unit 123, and a position control unit 124.

The kinematics computing unit 121 is a first computing device that computes the driving amount l_{k1b} of the wire 1b, based on the target bending angle θ_{1ref} of the bendable portion 112 that is input from the input device 130, and the distance between the wire 1b (predetermined wire) and wire 1a (other wire). Specifically, the kinematics computing unit 121 substitutes the target bending angle θ_{1ref} into the bending angle θ₁ in Expression (2) to calculate the driving amount l_{k1b} of the wire 1b.

The compensation amount computing unit 122 is a second computing device that computes the compensation amount l_{e1b} of the wire 1b, based on the target bending angle θ_{1ref} of the bendable portion 112 input from the input device 130, and the displacement Δl₁ₐ of the wire 1a. Specifically, the compensation amount computing unit 122 calculates the displacement Δl₁ₐ of the wire 1a using Expression (15), and calculates the compensation amount l_{e1b} of the wire 1b from the displacement Δl₁ₐ of the wire 1a using Expression (14).

The adding unit 123 adds the driving amount l_{k1b} calculated by the kinematics computing unit 121 and the compensation amount l_{e1b} calculated by the compensation amount computing unit 122 to calculate the drive control amount l_{p1b} for driving control of the wire 1b. That is to say, the adding unit 123 performs processing of compensating the driving amount l_{k1b} calculated by the kinematics computing unit 121 by the compensation amount l_{e1b} calculated by the compensation amount computing unit 122 to calculate the drive control amount l_{p1b} of the wire 1b. The position control unit 124 outputs a drive command τ_{1b} based on the drive control amount l_{p1b} to the continuum robot 110 (or more specifically, to the actuator 113). The adding unit 123 and position control unit 124 here make up a setting device that sets the drive control amount l_{p1b} for driving control amount of the wire 1b in the present embodiment.

### (1-3. Simulation)

Simulation is performed using the elastic wire driving model derived in the section 1-1 above, and the control system demonstrated in 1-2 above. The length l₀ of the bendable portion 112 is 0.01 m in the present embodiment. Angle error of the bendable portion 112 can be reduced in an arrangement where the total length (wire length) L₀ of the wire 1a and wire 1b is 1 m, and the control system according to the present embodiment is applied, which will be described below.

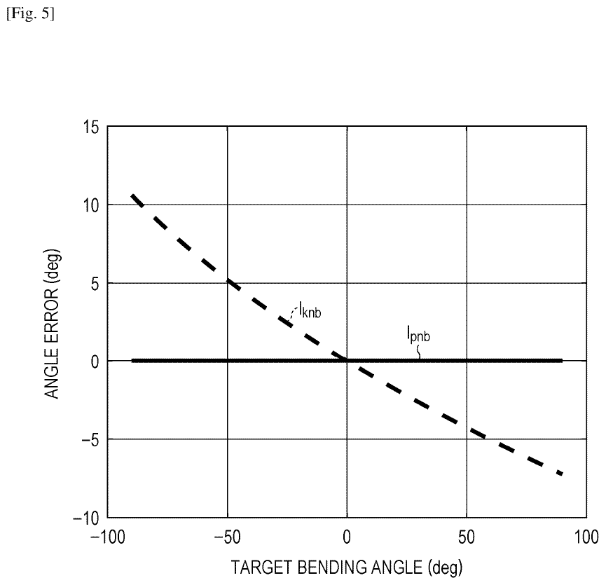

FIG. 5 is a characteristics diagram illustrating the relation between target bending angle and angle error according to the first embodiment of the present invention. The horizontal axis is the target bending angle (in degrees) in the characteristic diagram in FIG. 5, and the vertical axis is the angle error (in degrees).

Specifically, FIG. 5 illustrates angle error at the time of changing the target bending angle θ_{1ref} from −90 degrees to 90 degrees. The solid line in FIG. 5 illustrates angle error of response where the drive control amount l_{p1b} of the wire 1b computed using the elastic wire driving model has been input, and the dashed line illustrates the angle error where the driving amount l_{k1b} of the wire 1b computed using the kinematics model is input. According to the characteristics illustrated in FIG. 5, it can be seen that in a case where the drive control amount l_{p1b} according to the present embodiment is used, the angle error is constantly 0 even if the target bending angle θ_{1ref} changes. On the other hand, it can be seen that the angle error increases or decreases if the target bending angle θ_{1ref} increases in a case of using the driving amount l_{k1b} according to a comparative example. Thus, it can be seen that error in the attitude of the continuum robot 110 can be reduced by applying the control system according to the present embodiment, illustrated in FIG. 4.

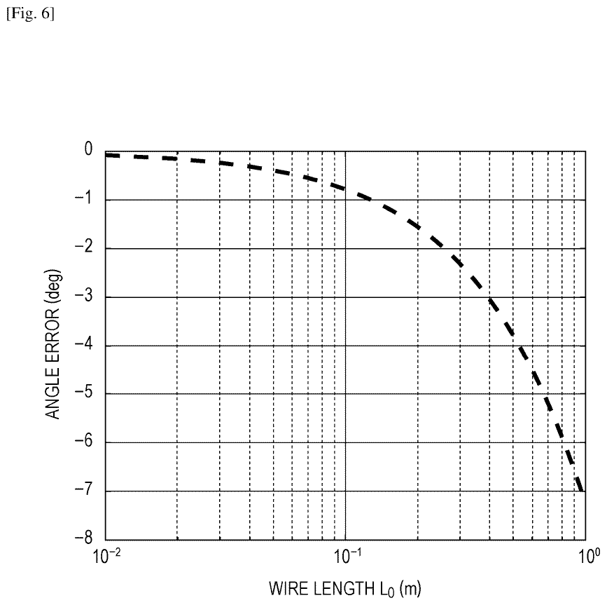

Next, description will be made to demonstrate that the control system according to the present embodiment illustrated in FIG. 4 is particularly advantageous in attitude control of the continuum robot 110 that has wires of a length longer than the length of the bendable portion 112. FIG. 6 is a characteristics diagram illustrating the relationship between the wire length L₀ and angle error according to the first embodiment of the present invention. The horizontal axis in the characteristics diagram in FIG. 6 is the wire length L₀, and the vertical axis is angle error (in degrees).

Specifically, in the present embodiment where the length l₀ of the bendable portion 112 is 0.01 m, FIG. 6 illustrates the bending angle error in a case where driving amount l_{k1b} is input to the continuum robot 110 where the target bending angle θ_{1ref} is 90 degrees and the wire length L₀ is 0.01 m to 1 m. It can be seen from the characteristics illustrated in FIG. 6 that great angle error occurs in a case where the wire length L₀ is 0.1 m or longer. Accordingly, the control system according to the present embodiment illustrated in FIG. 4 is particularly advantageous in the continuum robot 110 where the wire length L₀ is longer than the length l₀ of the bendable portion 112 by ten times or more.

The continuum robot control device 120 according to the first embodiment is configured to compute the compensation amount l_{e1b} of the wire 1b at the compensation amount computing unit 122, based on the target bending angle θ_{1ref} of the bendable portion 112 input from the input device 130, and the displacement Δl₁ₐ of the wire 1a. The adding unit 123 and the position control unit 124 perform processing of compensating the driving amount l_{k1b} calculated at the kinematics computing unit 121 by the compensation amount l_{e1b} calculated by the compensation amount computing unit 122, thereby setting the drive control amount l_{p1b} of the wire 1b.

According to this configuration, error in the driving amount l_{k1b} due to wire deformation can be compensated. Thus, error between the target posture of the bendable portion 112 and the actual attitude can be reduced, and as a result, reduction in the risk of contact between a path in the body or the like and the bendable portion can be realized.

Although the compensation amount computing unit 122 derives the compensation amount l_{e1b} using the displacement Δl₁ₐ of the wire 1a (other wire) in the present embodiment, an arrangement may be made where the compensation amount l_{e1b} is derived using the displacement Δl_{1b} of the wire 1b (predetermined wire). Specifically, the following Expression (18) is derived where Δl₁ₐ in Expression (15) is replaced by −Δl_{1b} using Expression (13), and Δl_{1b} is calculated using Expression (18). Expression (19) where Δl₁ₐ in Expression (14) has been replaced by −Δl_{1b} is then used to compute the compensation amount l_{e1b}.

$$\begin{matrix}[ Math \text{.5} ] & \\ \frac{- {A}_{3} ( {θ}_{1} ) Δ {l}_{1 b}^{3} + {A}_{2} ( {θ}_{1} ) Δ {l}_{1 b}^{2} - {A}_{1} ( {θ}_{1} ) Δ {l}_{1 b} + {A}_{0} ( {θ}_{1} )}{Δ {l}_{1 b}^{2} - {B}_{1} ( {θ}_{1} ) Δ {l}_{1 b} + {B}_{0} ( {θ}_{1} )} = 0 & ( 18 )\end{matrix}$$

$${l}_{e 1 b} = - 2 Δ {l}_{1 b} \qquad ( 19 )$$

Although only a method of computing compensation amount from the displacement of the other wire is illustrated in the following embodiments, the compensation amount can be computed using the displacement of the predetermined wire, in the same way as in the present embodiment. Second Embodiment

Next, a second embodiment of the present invention will be described. The second embodiment is an arrangement of applying a continuum robot having multiple (specifically, two) bendable portions in the direction of inserting and removing to and from the path.

Expression (15) according to the first embodiment is a cubic polynomial regarding wire displacement, so an analytical solution can be easily found. However, the highest order of displacement in the polynomial corresponding to Expression (15) is the fifth order or higher for continuum robots having multiple bendable portions, so finding an analytical solution is difficult. Accordingly, the control system according to the second embodiment finds a numerical solution for displacement by iterative calculation. Accordingly, the control system according to the present invention can be applied to a continuum robot that has multiple bendable portions. In the iterative calculation, numerical values are sequentially substituted into multiple equations derived by the elastic wire driving model, and wire displacement is updated. Accordingly, iterative calculation can be performed by combining simple update rules even if the number of bendable portions increases.

In the following, first, a kinematics model and elastic wire driving model for a continuum robot will be derived. Thereafter, the control system according to the present embodiment where wire displacement is calculated using iterative calculation will be demonstrated, and the capability to improve control performance even in a continuum robot having multiple bendable portions will be described by way of numerical examples.

### (2-1. Modelling)

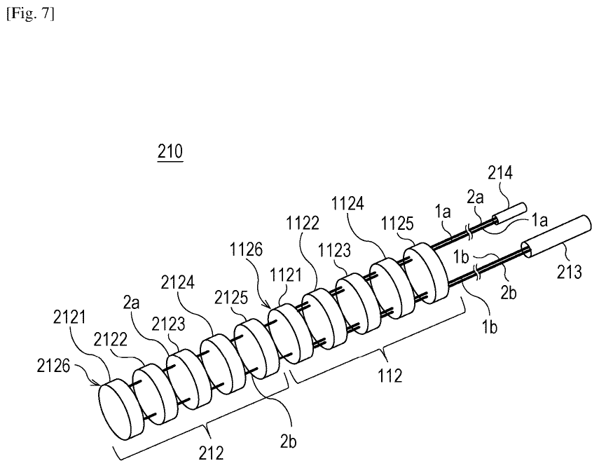

FIG. 7 is a diagram illustrating an example of a schematic configuration of a continuum robot 210 used in the second embodiment of the present invention. Configurations that are the same as configurations illustrated in FIGS. 1 and 2 are denoted by the same symbols, and detailed description thereof will be omitted. A path into which bendable portions 112 and 212 are inserted and removed from is omitted from illustration in FIG. 7.

Specifically, the continuum robot 210 illustrated in FIG. 7 is configured having a first bendable portion 112 that is equivalent to the bendable portion 112 illustrated in FIG. 2, and a second bendable portion 212. That is to say, the continuum robot 210 illustrated in FIG. 7 has multiple bendable portions 112 and 212 serially arranged.

The second bendable portion 212 has multiple wires 2a and 2b extending through a face at the proximal end of the second bendable portion 212 (the face at the distal end 1126 of the first bendable portion 112), a first wire guide 2121 to which the multiple wires 2a and 2b are fixed at different positions, that guides the multiple wires 2a and 2b, and second wire guides 2122 through 2125 that are disposed between the face at the proximal end described above and the first wire guide 2121, and that guide the multiple wires 2a and 2b.

Holes are provided to the wire guides 1121 through 1125 of the first bendable portion 112 on the outer side of the guide holes for the wire 1a and wire 1b, for example, for guiding the wire 2a and wire 2b. The wires are guided through the elongated portion 111 in the same way as with the continuum robot 110 in the first embodiment, with the wire 1a and wire 2a being connected to a fixing portion 214, and the wire 1b and wire 2b being connected to an actuator 213. In the example illustrated in FIG. 7, the actuator 213 is a driving mechanism where the wire 1b and wire 2b can each be independently driven, and an arrangement may be made in the present embodiment where two actuators 213 are provided corresponding to the wire 1b and wire 2b. Accordingly, the first bendable portion 112 can be driven by driving the wire 1b and the second bendable portion 212 can be driven by driving the wire 2b in the present embodiment. In the second embodiment described below, the following Assumptions 10 through 12 are added to the above-described Assumptions 1 through 9, and a kinematic model and elastic wire driving model in the continuum robot 210 according to the second embodiment are derived. Assumption 10 is that the distance between the guide hole for the wire 1a and the guide hole for the wire 2a is short, so the difference between each guide hole and the center axis of the bendable portion is R_{g}/2 for both. This is the same regarding the relation between the guide hole for the wire 1b and the guide hole for the wire 2b using the same method as with the first embodiment described above. Assumption 11 is that the length l₂ₐ₁ of the wire 2a and the length l₁ₐ₁ of the wire 1a are the same in the first bendable portion 112. This is the same regarding the length l_{2b1} of the wire 2b and the length l_{1b1} of the wire 1b as well. Assumption 12 is that only bending moment of the wires acts between the distal end 1126 of the first bendable portion 112 and the wire 2a and wire 2b, and that there is no friction or reactive force acting.

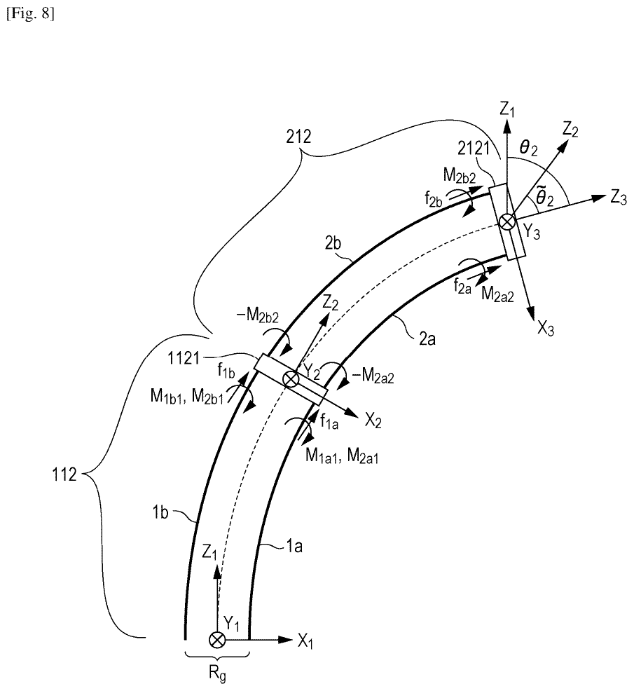

FIG. 8 is a diagram illustrating an example of a kinematic model at the first bendable portion 112 and second bendable portion 212 illustrated in FIG. 7, according to the second embodiment of the present invention. In FIG. 8, a center position of the plane at the distal end 2126 of the second bendable portion 212, illustrated in FIG. 7, is the origin. The normal direction on the plane at the distal end 2126 is represented by a Z₃ axis, the circumferential direction by an X₃ axis, and the direction from the plane of the drawing toward the far side by a Y₃ axis. The angle between the Z axis and the Z₃ axis is an absolute bending angle θ₂, and an angle between the Z axis and the Z₃ axis is a relative bending angle tildeθ₂, as illustrated in FIG. 8. This bending angle tildeθ₂ is indicated by a tilde indicating a relative coordinate system, since the reference face of the second bendable portion 212 differs from the reference face of the first bendable portion 112. Note that the notation in the following Expressions have a tilde superscript over the symbol θ indicating a relative coordinate system, but this has been written out in the text of the present specification as bending angle “tildeθ₂” or the like, which is true throughout the following description.

In the present embodiment, the relative bending angle tildeθ₂ is expressed by the following Expression (20) using bending angles θ₁ and θ₂.

$$\begin{matrix}[ Math \text{.6} ] & \\ = {θ}_{2} - {θ}_{1} & ( 20 )\end{matrix}$$

Also, the relative bending angle tildeθ₂ is expressed by the following Expression (21) using the length 1₂ₐ₂ of the wire 2a and the length l_{2b2} of the wire 2b in the second bendable portion 212, in the same way as Expression (1) in the first embodiment. [Math. 7]

$$\begin{matrix}[ Math \text{.7} ] & \\ {R}_{g} = {l}_{2 b 2} - {l}_{2 a 2} & ( 21 )\end{matrix}$$

Accordingly, the driving amount l_{k2b} of the second bendable portion 212 when assuming that the wires are rigid in the longitudinal direction can be expressed by the following Expression (22).

$$\begin{matrix}[ Math \text{.8} ] & \\ {l}_{k 2 b} = {R}_{g} & ( 22 )\end{matrix}$$

Also, Expression (2) holds at the first bendable portion 112 of the continuum robot 210 according to the present embodiment, so the kinematic model can be expressed using Expressions (2) and (22).

Taking into consideration stretching and contraction of the wires, the length of the wire 2a and wire 2b at the first bendable portion 112 and second bendable portion 212 can be expressed by the following Expressions (23) and (24), using the displacement Δ1₂ₐ of the wire 2a and the displacement Δ1_{2b} of the wire 2b, and the drive control amount l_{p2b} of the wire 2b. l₂ₐ₁+l₂ₐ₂=l₂₀+Δl₂ₐ (23) l_{2b1}+l_{2b2}=l₂₀+Δl_{2b}+l_{p2b} (24)

Now, the length l₁ₐ₁ and the length l₂ₐ₁, and the length l_{1b1} and the length l_{2b1}, are equal, from the above-described Assumption 11, so substituting Expression (3) into Expression (23) and substituting Expression (4) into Expression (24) enables the lengths l₂ₐ₂ and l_{2b2} to be respectively expressed by the following Expressions (25) and (26).

$${l}_{2 a 2} = {l}_{20} - {l}_{10} + Δ {l}_{2 a} - Δ {l}_{1 a} \qquad ( 25 )$$

$${l}_{2 b 2} = {l}_{20} - {l}_{10} + Δ {l}_{2 b} - Δ {l}_{1 b} + {l}_{p 2 b} - {l}_{p 1 b} \qquad ( 26 )$$

Substituting Expressions (22), (25), and (26) into Expression (21), and further eliminating the drive control amount l_{p1b} using Expression (5) yields the following Expression (27).

$${1}_{p 2 b} = {1}_{k 2 b} + {1}_{k 1 b} + ( Δ {l}_{2 a} - Δ {l}_{2 b} ) \qquad ( 27 )$$

Accordingly, obtaining the compensation amount l_{e2b} of the wire 2b at the second bendable portion 212 by the following Expression (28) in the same way as in Expression (6) in the first embodiment enables the error in the driving amount due to stretching and contraction of the wires at the second bendable portion 212 to be compensated.

$${l}_{e 2 b} = Δ {l}_{2 a} - Δ {l}_{2 b} \qquad ( 28 )$$

Note that as described above, the kinematics of the first bendable portion 112 in the second embodiment are the same as in the first embodiment described above, so the drive control amount l_{p1b} and compensation amount l_{e1b} of the wire 1b are expressed by Expressions (5) and (6) in the same way as in the first embodiment, respectively.

An expression of balance of the force and moment acting on the distal end 1126 of the first bendable portion 112 and the distal end 2126 of the second bendable portion 212 is derived, to obtain the wire displacement. The balance of moment on the Y₃ axis that acts on the distal end 2126 of the second bendable portion 212 is expressed by the following Expression (29), in the same way as in Expression (8) in the first embodiment.

$${M}_{2 a 2} + {M}_{2 b 2} - {R}_{g} {f}_{2 a} = 0 \qquad ( 29 )$$

Balance in the Z₃ axial direction is expressed by the following Expression (30).

$${f}_{2 a} + {f}_{2 b} = 0$$

Also, moments M₂ₐ₂ and M_{2b2}, and forces f₂ₐ and f_{2b}, can be expressed by the following Expressions (31) and (32), in the same way as in Expressions (10) and (9), respectively.

$$\begin{matrix}[ Math \text{.9} ] & \\ {M}_{2 a 2} = - \frac{{k}_{m}}{{l}_{2 a 2}} , & ( 31 )\end{matrix}$$

$${M}_{2 b 2} = - \frac{{k}_{m}}{{l}_{2 b 2}}$$

$${f}_{2 a} = - {k}_{e} Δ {l}_{2 a} , \qquad ( 32 )$$

$${f}_{2 b} = - {k}_{e} Δ {l}_{2 b}$$

The following Expression (33) then holds, from Expressions (30) and (32).

$$Δ {l}_{2 b} = - Δ {l}_{2 a} \qquad ( 33 )$$

Accordingly, substituting Expression (33) into Expression (28) yields the following Expression (34).

l_{e2b}=2Δ1₂ₐ (34)

It can thus be seen from Expressions (14) and (34) that once the displacement Δl₁ₐ of the wire 1a and the displacement Δl₂ₐ of the wire 2a have been found, the compensation amount l_{e1b} of the wire 1a and the compensation amount l_{e2b} of the wire 2a can be computed. In addition to the bending moment of the wire 1a, wire 1b, wire 2a, and wire 2b at the first bendable portion 112, anti-moment of moments M₂ₐ₂ and M_{2b2} act on the distal end 1126 of the bendable portion 112, based on the above-described Assumption 12. Accordingly, the balance of moment on the Y₂ axis at the distal end 1126 of the first bendable portion 112 can be expressed by the following Expression (35).

$$( {M}_{1 a 1} + {M}_{1 b 1} + {M}_{2 a 1} + {M}_{2 b 1} ) - ( {M}_{2 a 2} + {M}_{2 b 2} ) - {R}_{g} {f}_{1 a} = 0 \qquad ( 35 )$$

From the above-described Assumption 11, the wire length l₁ₐ₁ and the wire length l₂ₐ₁ are the same, and the wire length l_{1b1} and the wire length l_{2b1} are the same. Accordingly, moment M₂ₐ₁ and moment M₁ₐ₁ are equal, moment M_{2b1} and moment M_{1b1} are equal, and the following Expression (36) holds.

$${M}_{1 a 1} = {M}_{2 a 1} , {M}_{1 b 1} = {M}_{2 b 1} \qquad ( 36 )$$

Accordingly, Expression (35) can be modified to the following Expression (37).

$$2 ( {M}_{1 a 1} + {M}_{1 b 1} ) - ( {M}_{2 a 2} + {M}_{2 b 2} ) - {R}_{g} {f}_{1 a} = 0 \qquad ( 37 )$$

Only tension of the wire 1a and wire 1b act on the Z₂ axial direction of the distal end 1126 of the first bendable portion 112, from the above-described Assumption 12, so the Expression of balance of force for the first bendable portion 112 is expressed by Expression (7), in the same way as in the first embodiment.

### (2-2. Control System)

Performing modification of Expressions the same as with the first embodiment on the elastic wire driving model according to the second embodiment enables two independent polynomials to be obtained, where the displacement Δl₁ₐ of the wire 1a and the displacement Δl₂ₐ of the wire 2a are variables. However, these are high-order polynomials of five or more orders regarding the displacement Δl₁ₐ and Δl₂ₐ, so finding an analytical solution is difficult. At this time, a numerical solution can be obtained by an optimization method such as the gradient method or the like, but deriving a highorder polynomial regarding the continuum robot 210 having a great number of bendable portions is not easy. Accordingly, in the second embodiment, numerical solutions are found for the displacement Δl₁ₐ of the wire 1a and the displacement Δl₂ₐ of the wire 2a by iterative calculation where simple expressions derived from the elastic wire driving model are combined.

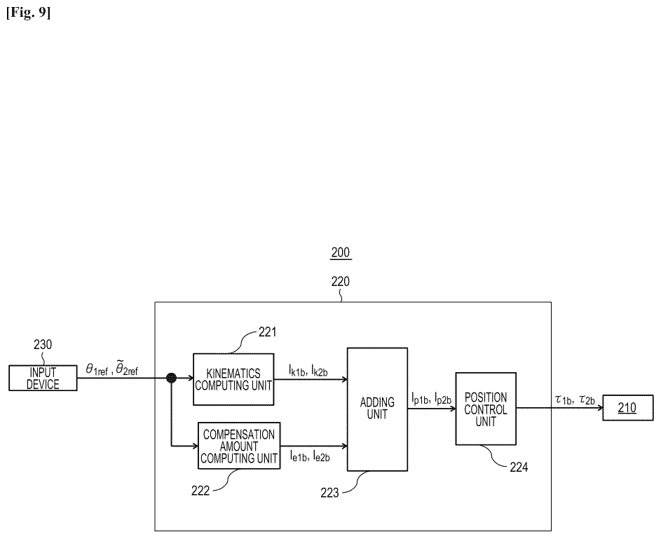

FIG. 9 is a diagram illustrating an example of the functional configuration of a continuum robot control system 200 according to the second embodiment of the present invention. The continuum robot control system 200 illustrated in FIG. 9 is configured including the continuum robot 210 illustrated in FIG. 7, a continuum robot control device 220, and an input device 230.

In the second embodiment, the input device 230 inputs, in addition to the target bending angle θ_{1ref} of the first bendable portion 112, a target bending angle (reference relative bending angle tildeθ_{2ref}), to the continuum robot control device 220.

The continuum robot control device 220 sets an drive control amount l_{p1b} for driving control of the wire 1b, and an drive control amount l_{p2b} for driving control of the wire 2b, based on a target bending angle θ_{1ref} for the first bendable portion 112 and a target bending angle tildeθ_{2ref} for the second bendable portion 212, input from the input device 230, and outputs a drive command τ_{1b} based on the drive control amount l_{p1b} and a drive command τ_{2b} based on the drive control amount l_{p2b}, to the continuum robot 210 (or more specifically, to the actuator 213).

The continuum robot control device 220 illustrated in FIG. 9 is configured including a kinematics computing unit 221, a compensation amount computing unit 222, an adding unit 223, and a position control unit 224.

The kinematics computing unit 221 computes the driving amount l_{k1b} of the wire 1b, based on the target bending angle θ_{1ref} of the first bendable portion 112 input from the input device 230 and the distance between the wire 1b (predetermined wire) and wire 1a (other wire), and also computes the driving amount l_{k2b} of the wire 2b, based on the target bending angle tildeθ_{2ref} of the second bendable portion 212 input from the input device 230 and the distance between the wire 2b (predetermined wire) and wire 2a (other wire). Specifically, the kinematics computing unit 221 substitutes the target bending angle θ_{1ref} into the bending angle θ₁ in Expression (2) to calculate the driving amount l_{k1b} of the wire 1b, and also substitutes the target bending angle tildeθ_{2ref} into the bending angle tildeθ₂ in Expression (22) to calculate the driving amount l_{k2b} of the wire 2b.

The compensation amount computing unit 222 is a second computing device that computes the compensation amount l_{e1b} of the wire 1b, based on the target bending angle θ_{1ref} of the first bendable portion 112 input from the input device 230, and the displacement Δl₁ₐ of the wire 1a, and that computes the compensation amount l_{e2b} of the wire 2b, based on the target bending angle tildeθ_{2ref} of the second bendable portion 212 input from the input device 230, and the displacement Δl₂ₐ of the wire 2a. Specifically, the compensation amount computing unit 222 calculates the displacement Δl₁ₐ of the wire 1a using Expression (15), and calculates the compensation amount l_{e1b} of the wire 1b from the displacement Δl₁ₐ of the wire 1a using Expression (14). The compensation amount computing unit 222 also calculates the displacement Δl₂ₐ of the wire 2a in the same way, and calculates the compensation amount l_{e2b} of the wire 2b from the displacement Δl₂ₐ of the wire 2a using Expression (34).

The adding unit 223 adds the driving amount l_{k1b} calculated by the kinematics computing unit 221 and the compensation amount l_{e1b} calculated by the compensation amount computing unit 222 to calculate the drive control amount l_{p1b} for driving control of the wire 1b. The adding unit 223 also adds the driving amount l_{k2b} calculated by the kinematics computing unit 221 and the compensation amount l_{e2b} calculated by the compensation amount computing unit 222 to calculate the drive control amount l_{p2b} for driving control of the wire 2b. That is to say, the adding unit 223 performs processing of compensating the driving amount l_{k1b} and l_{k2b} calculated by the kinematics computing unit 221, respectively by the compensation amounts l_{e1b} and l_{e2b} calculated by the compensation amount computing unit 222 to calculate the drive control amount l_{p1b} of the wire 1b and the drive control amount l_{p2b} of the wire 2b. The position control unit 224 outputs a drive command τ_{1b} based on the drive control amount l_{p1b} and a drive command To based on the drive control amount l_{p2b} to the continuum robot 210 (or more specifically, to the actuator 213). In the present embodiment, the adding unit 223 and position control unit 224 make up a setting device that sets the drive control amount l_{p1b} for driving control of the wire 1b and the drive control amount l_{p1b} for driving control of the wire 2b.

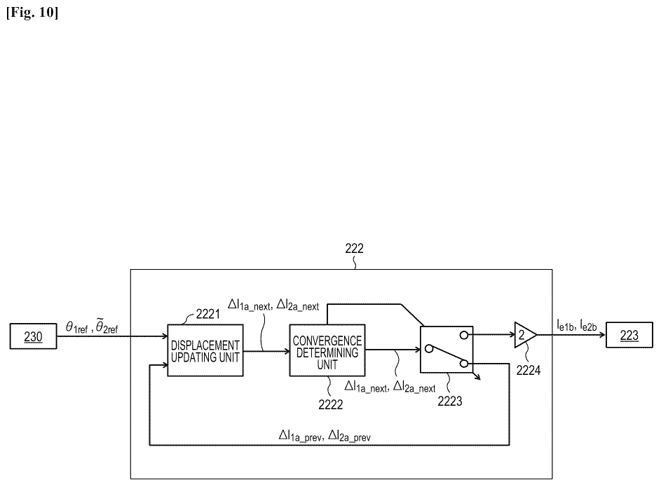

FIG. 10 is a diagram illustrating an example of the functional configuration of the compensation amount computing unit 222 illustrated in FIG. 9, according to the second embodiment of the present invention. The compensation amount computing unit 222 is configured including a displacement updating unit 2221, a convergence determining unit 2222, an output switching unit 2223, and an amplifying unit 2224, as illustrated in FIG. 10.

In accordance with input of the target bending angles θ_{1ref} and tildeθ_{2ref}, and first displacement candidate values Δl_{1a_prev} and Δl_{2a_prev} for the displacements of the wires 1a and 2b the displacement updating unit 2221 updates to second displacement candidate values Δl_{1a_next} and Δl_{2a_next}. At this time, the displacement updating unit 2221 updates the second displacement candidate values Δl_{1a_next} and Δl_{2a_next} of the displacement of the wires 1a and 2b using the elastic wire driving model described above.

The convergence determining unit 2222 determines whether or not the second displacement candidate values Δl_{1a_next} and Δl_{2a_next} have converged at a constant value. In a case of having determined that the second displacement candidate values Δl_{1a_next} and Δl_{2a_next} have converged at a constant value, the convergence determining unit 2222 causes the output switching unit 2223 to output the second displacement candidate values Δl_{1a_next} and Δl_{2a_next} respectively as displacement Δl₁ₐ and displacement Δl₂ₐ to the amplifying unit 2224. In a case of having determined that the second displacement candidate values Δl_{1a_next} and Δl_{2a_next} have not converged at a constant value, the convergence determining unit 2222 causes the output switching unit 2223 to output the second displacement candidate values Δl_{1a_next} and Δl_{2a_next} as first displacement candidate values Δl_{1a_prev} and Δl_{2a_prev} to the displacement updating unit 2221, and iterative calculation is performed again. The convergence determining unit 2222 and output switching unit 2223 according to the present embodiment make up a configuration corresponding to an output processing unit according to the present invention.

The amplifying unit 2224 is a compensation amount calculating unit that uses the second displacement candidate values Δl_{1a_next} and Δl_{2a_next} as displacement Δl₁ₐ and displacement Δl₂ₐ respectively, to calculate the compensation amounts l_{e1b} and l_{e2b}. The compensation amounts l_{e1b} and l_{e2b} are each double the displacement Δl₁ₐ and displacement Δl_{1b} respectively in the present embodiment, as illustrated in in Expressions (14) and (34). Accordingly, the amplifying unit 2224 multiples the displacement Δl₁ₐ by 2 with regard to the first bendable portion 112 and calculates the compensation amount l_{e1b}, and multiples the displacement Δl₂ₐ by 2 with regard to the second bendable portion 212 and calculates the compensation amount l_{e2b}.

Note that an arrangement may be made where, in a case that the second displacement candidate values Δl_{1a_next} and Δl_{2a_next} do not converge at a constant value within a predetermined iteration count, the convergence determining unit 2222 causes the output switching unit 2223 to output the last second displacement candidate values Δl_{1a_next} and Δl_{2a_next} to have converged, respectively as displacement Δl₁ₐ and displacement Δl₂ₐ to the amplifying unit 2224. This can prevent the compensation amounts calculated at the amplifying unit 2224 from changing discontinuously. Although the initial values of the first displacement candidate values Δl_{1a_prev} and Δl_{2a_prev} in the iterative calculation in the present embodiment are 0, the displacements that converged last may be used as the initial values.

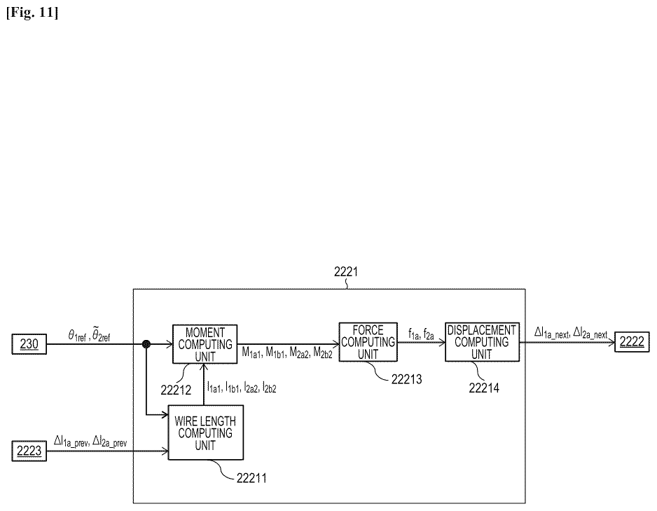

FIG. 11 is a diagram illustrating an example of the functional configuration of the displacement updating unit 2221 illustrated in FIG. 10 according to the second embodiment of the present invention. The displacement updating unit 2221 is configured including a wire length computing unit 22211, a moment computing unit 22212, a force computing unit 22213, and a displacement computing unit 22214, as illustrated in FIG. 11.

The wire length computing unit 22211 calculates the wire lengths l₁ₐ₁, l_{1b1}, l₂ₐ₂, and l_{2b2}, for each of the multiple wires 1a and 1b and wires 2a and 2b at the first bendable portion 112 and second bendable portion 212, in accordance with the input of the target bending angles θ_{1ref} and tildeθ_{2ref} and first displacement candidate values Δl_{1a_prev} and Δl_{2a_prev}. Specifically, the wire length computing unit 22211 calculates the wire lengths l₁ₐ₁, l_{1b1}, l₂ₐ₂, and l_{2b2}, using the following Expressions (38) through (41), where Expressions (1) through (5) and Expressions (21) through (27) have been modified.

$$\begin{matrix}[ Math . 10 ] & \\ {l}_{1 a 1} = {l}_{10} + Δ {l}_{1 a \_ prev} & ( 38 )\end{matrix}$$

$${l}_{1 b 1} = {l}_{10} + Δ {l}_{1 a \_ prev} + {R}_{g} {θ}_{1 ref} \qquad ( 39 )$$

$${l}_{2 a 2} = {l}_{20} - {l}_{10} + Δ {l}_{2 a \_ prev} - Δ {l}_{1 a \_ prev} \qquad ( 40 )$$

$${l}_{2 b 2} = {l}_{20} - {l}_{10} + Δ {l}_{2 a \_ prev} - Δ {l}_{1 a \_ prev} + {R}_{g} {\tilde{θ}}_{2 ref} \qquad ( 41 )$$

The moment computing unit 22212 calculates bending moments M₁ₐ₁, M_{1b1}, M₂ₐ₂, and M_{2b2}, for each of the multiple wires 1a and 1b and wires 2a and 2b, in accordance with input of the target bending angles θ_{1ref} and tildeθ_{2ref} and the wire lengths l₁ₐ₁, l_{1b1}, l₂ₐ₂, and l_{2b2}. Specifically, the moment computing unit 22212 calculates the bending moments M₁ₐ₁, M_{1b1}, M₂ₐ₂, and M_{2b2} using Expressions (10) and (36).

The force computing unit 22213 calculates forces f₁ₐ and f₂ₐ representing the tensile force on the wires 1a and 2a (other wires), in accordance with the input of the bending moments M₁ₐ₁, M_{1b1}, M₂ₐ₂, and M_{2b2} of the multiple wires 1a and 1b and wires 2a and 2b. Specifically, the force computing unit 22213 calculates the forces f₁ₐ and f₂ₐ using Expressions (29) and (37).

The displacement computing unit 22214 computes the second displacement candidate values Δl_{1a_next} and Δl_{2a_next} in accordance with input of the forces f₁ₐ and f₂ₐ, and updates displacement candidate values. Specifically, the displacement computing unit 22214 uses Expressions (9) and (32) to compute the second displacement candidate values Δl_{1a_next} and Δl_{2a_next}, and updates the displacement candidate values. Accordingly, the second displacement candidate values Δl_{1a_next} and Δl_{2a_next} after updating in the present embodiment are represented by the following Expressions (42) and (43), respectively.

$$\begin{matrix}[ Math . 11 ] & \\ Δ {l}_{1 a \_ next} = - \frac{{k}_{m}}{{R}_{g} {k}_{e}} \{ 2 ( \frac{1}{{l}_{10} + {Δ}_{1 a \_ prev} + {R}_{g} {θ}_{1 ref}} + \frac{1}{{l}_{10} + {Δ}_{1 a \_ prev}} ) {θ}_{1 ref} + ( \frac{1}{{l}_{20} - {l}_{10} + {Δ}_{2 a \_ prev} - {Δ}_{1 a \_ prev} + {R}_{g} {\tilde{θ}}_{2 ref}} + \frac{1}{{l}_{20} - {l}_{10} + {Δ}_{2 a \_ prev} - {Δ}_{1 a \_ prev}} ) {\tilde{θ}}_{ref} \} & ( 42 )\end{matrix}$$

$$Δ {l}_{2 a \_ next} = \frac{{k}_{m}}{{R}_{g} {k}_{e}} ( \frac{1}{{l}_{20} - {l}_{10} + {Δ}_{2 a \_ prev} - {Δ}_{1 a \_ prev} + {R}_{g} {\tilde{θ}}_{2 ref}} + \frac{1}{{l}_{20} - {l}_{10} + {Δ}_{2 a \_ prev} - {Δ}_{1 a \_ prev}} ) {\tilde{θ}}_{2 ref} \qquad ( 43 )$$

Note that Expressions (42) and (43) are illustrated to describe update rules, and that the displacement updating unit 2221 updates the displacement by sequentially substituting numerical values into multiple equations derived from the elastic wire driving model, as described above. Although the control system illustrated in FIGS. 9 through 11 according to the present embodiment perform convergence determination in iterative calculations using displacement, but wire length, force, and bending moment are also updated with each iterative calculation, so one of these may be used for determination of convergence.

Also, while the relative bending angle tildeθ_{2ref} is used in the present embodiment as the target bending angle of the second bendable portion 212, the absolute bending angle may be used as the target bending angle, since the absolute angle and relative angle are mutually convertible, as illustrated in Expression (20).

### (2-3. Simulation)

The control system according to the second embodiment, illustrated in FIGS. 9 through 11, is applied to the continuum robot 210 having the two bendable portions 112 and 212. Note that the lengths of the first bendable portion 112 and the second bendable portion 212 are 0.01 m, and the length of the elongated portion 111 is 1 m.

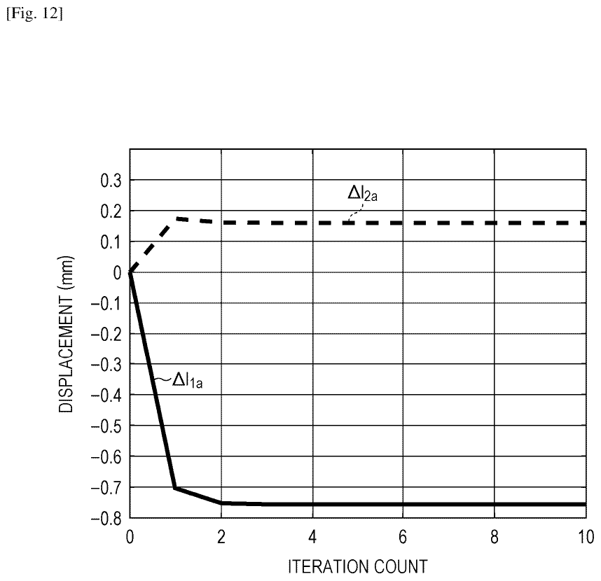

FIG. 12 is a characteristics diagram illustrating the relation between iteration count, which is a count of iterative calculations performed, and displacement, according to the second embodiment of the present invention. The horizontal axis in the characteristics diagram of FIG. 12 is the iteration count, and the vertical axis is the displacement in each iterative calculation performed.

Specifically, FIG. 12 shows the response of displacements Δl₁ₐ and Δl₂ₐ at the compensation amount computing unit 222 in a case where the target bending angle θ_{1ref} is 90 degrees and the target bending angle tildeθ_{2ref} is 90 degrees, and the initial values of the displacements Δl₁ₐ and Δl₂ₐ are 0. Also, the solid line in FIG. 12 represents the displacement Δl₁ₐ, and the dashed line represents the displacement Δl₂ₐ. It can be seen from FIG. 12 that the displacements Δl₁ₐ and Δl₂ₐ converge by iterative calculations, since they both become constant after the fourth calculation having been performed.

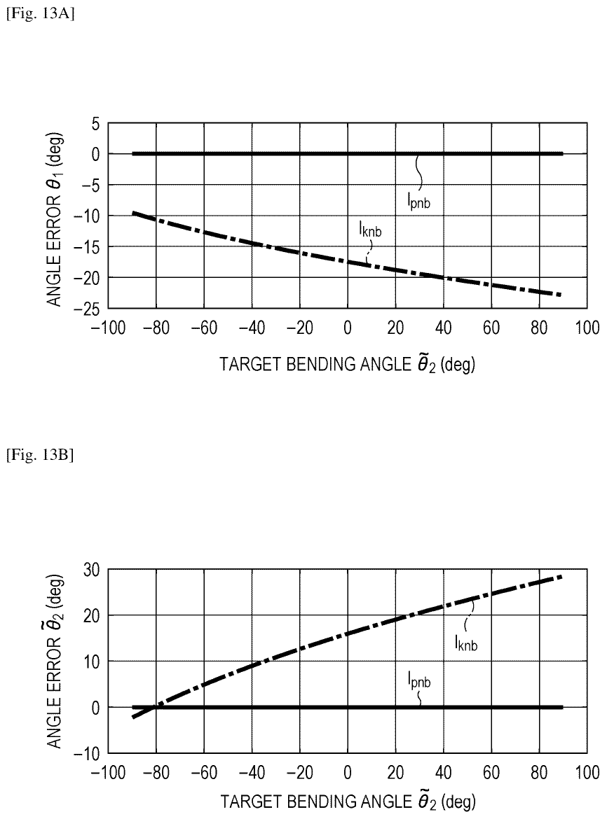

FIGS. 13A and 13B are characteristics diagrams illustrating the relation between target bending angle and angle error at the first bendable portion 112 and second bendable portion 212 according to the second embodiment of the present invention.

Specifically, FIG. 13A illustrates the angle error of the first bendable portion 112 in a case where the target bending angle θ_{1ref} is set to −90 degrees, and the target bending angle tildeθ_{2ref} is changed from −90 degrees to 90 degrees. Also, FIG. 13B illustrates the angle error of the second bendable portion 212 in a case where the target bending angle θ_{1ref} is set to −90 degrees, and the target bending angle tildeθ_{2ref} is changed from −90 degrees to 90 degrees. The horizontal axis is the target bending angle tildeθ_{2ref} in the characteristics diagrams of FIGS. 13A and 13B, while the vertical axis is angle error θ₁ in FIG. 13A and is angle error tildeθ₂ in FIG. 13B.

The dotted lines in FIGS. 13A and 13B respectively represent response as to input of the driving amounts l_{k1b} and l_{k2b} computed by the kinematics computing unit 221. The solid lines in FIGS. 13A and 13B respectively represent response as to input of the drive control amounts l_{p1b} and l_{p2b} computed from the elastic wire driving model according to the second embodiment.

It can be seen from FIGS. 13A and 13B that in a case of using the driving amounts l_{k1b} and l_{k2b} according to a comparative example, the angle error of the first bendable portion 112 and the second bendable portion 212 both increase along with increase or decrease of the target bending angle tildeθ_{2ref}. On the other hand, in a case of using the drive control amounts l_{p1b} and l_{p2b} according to the present embodiment, the angle error of the first bendable portion 112 and the second bendable portion 212 is constantly 0 even if the target bending angle tildeθ_{2ref} changes. Accordingly, it can be understood that applying the control system illustrated in FIGS. 9 through 11 according to the present embodiment enables attitude error of the continuum robot 210 that has multiple bendable portions to be reduced.

In the continuum robot control device 220 according to the second embodiment, the compensation amount computing unit 222 computes the respective compensation amounts 1_{e1b} and l_{e2b} for the wires 1b and 2b, based on the target bending angles θ_{1ref} and tildeθ_{2ref} that have been input from the input device 230, and the displacements Δl₁ₐ and Δl₂ₐ of the wires 1a and 2a, for each bendable portion of the multiple bendable portions 112 and 212. The adding unit 223 and position control unit 224 perform processing to compensate the respective driving amounts l_{k1b} and k_{k2b} calculated at the kinematics computing unit 221 by the compensation amounts l_{e1b} and l_{e2b} calculated at the compensation amount computing unit 222, thereby setting the drive control amounts l_{p1b} and l_{p2b} of the wires 1b and 2b.

According to this configuration, error in the driving amounts l_{k1b} and k_{k2b} due to wire deformation can be compensated for in the continuum robot 210 having multiple bendable portions, as well. Accordingly, error between the reference position of the multiple bendable portions and the actual attitude can be reduced, and as a result, reduction in the risk of contact between a path in the body or the like and the bendable portion can be realized.

Note that the driving amount of each wire can be compensated for in the present embodiment using the compensation amount calculated based on the displacement of any one wire of the multiple wires relating to the same bendable portion, in the same way as in the first embodiment.

### Third Embodiment

Next, a third embodiment of the present invention will be described. The third embodiment is an arrangement of applying a continuum robot having a bendable portion that is bendable three-dimensionally. Specifically, in the continuum robot according to the third embodiment, one bendable portion is bent by controlling driving of two wires, and the control system computes compensation amounts taking into consideration the displacement of these two wires, by iterative calculation similar to that in the second embodiment. Accordingly, the control capabilities of the continuum robot having a bendable portion that is bendable three-dimensionally can be improved.

### (3-1. Modelling)

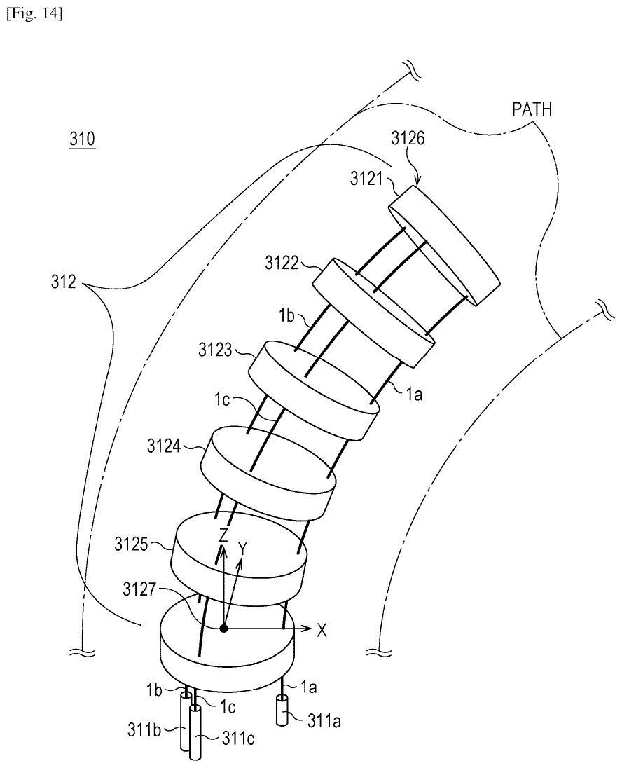

FIG. 14 is a diagram illustrating an example of a schematic configuration of a continuum robot 310 used in the third embodiment of the present invention. Configurations that are the same as configurations illustrated in FIGS. 1, 2, and 7 are denoted by the same symbols in FIG. 14, and detailed description thereof will be omitted. FIG. 14 also illustrates an example of a path into which a bendable portions 312 is inserted and removed from.

The bendable portion 312 has multiple wires 1a, 1b, and 1c extending through a face at the proximal end of the bendable portion 312 (the face where the center position is position 3127), a first wire guide 3121 to which the multiple wires 1a, 1b, and 1c are fixed at different positions, that guides the multiple wires 1a, 1b, and 1c, and second wire guides 3122 through 3125 that are disposed between the face at the proximal end described above and the first wire guide 3121, and that guide the multiple wires 1a, 1b, and 1c.

The wires are guided through the elongated portion 111 in the same way as with the continuum robot 110 according to the first embodiment, with the wire 1a being connected to a fixing portion 311a, the wire 1b being connected to an actuator 311b, and the wire 1c being connected to an actuator 311c. In the present embodiment, the wire 1a, wire 1b, and wire 1c are respectively laid out at the apices of an equilateral triangle of which the center axis of the bendable portion 312 is the center of gravity. Setting the drive control amount of the wire 1b and wire 1c enables the bendable portion 312 to be bent within an optional plane. For example, in a case where the wire 1b and wire 1c are driven in the same direction by the same amount, the bendable portion 312 bends within the X-Z plane in FIG. 14. In a case where the wire 1b and wire 1c are driven in opposites directions by the same amount, the bendable portion 312 bends within the Y-Z plane. A kinematic model and elastic wire driving model in the continuum robot 310 are derived in the present embodiment as well.

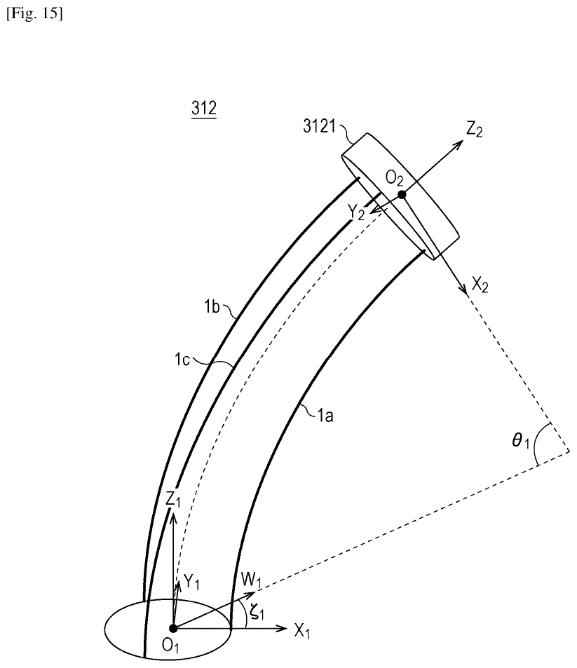

FIG. 15 is a diagram illustrating an example of a kinematic model at the bendable portion 312 illustrated in FIG. 14, according to the third embodiment of the present invention. In FIG. 15, the center position 3127 at the face of the proximal end of the bendable portion 312 illustrated in FIG. 14 is an origin O₁. The direction of the wire 1a is represented by an X₁ axis, the longitudinal direction of the bendable portion 312 is represented by a Z₁ axis, and a direction orthogonal to the X₁ axis and Z₁ axis is represented by a Y₁ axis. A direction from the origin O₁ in the X₁-Y₁ plane toward a center point O₂ in a face at the distal end 3126 of the bendable portion 312 is represented by a W₁ axis. And the angle formed between the X₁ axis and the W₁ axis is a rotation angle ζ₁. Further, a center axis at the distal end 3126 is the origin, the normal direction is represented by a Z₂ axis, an X₂ axis is perpendicular to the Z₂ axis and on a W₁-Z₁ plane, and a Y₂ axis is in a direction perpendicular to the X₂ axis and Z₂ axis.

When the bending angle is θ₁ and the rotation angle is ζ₁, and the wire 1a and wire 1b are projected onto the W₁-Z₁ plane, a distance R_{ba} between the wire 1a and wire 1b on the W₁-Z₁ plane can be expressed by the following Expression (44). [Math. 12]

$$\begin{matrix}[ Math . 12 ] & \\ {R}_{ba} = \frac{\sqrt{3}}{2} {R}_{g} cos ( - {ζ}_{1} + \frac{π}{6} ) & ( 44 )\end{matrix}$$

Accordingly, the following Expression (45) holds between the bending angle θ₁ and lengths l₁ₐ₁ and l_{1b1}, in the same way as in Expression (1) according to the first embodiment.

$$\begin{matrix}[ Math . 13 ] & \\ \frac{\sqrt{3}}{2} {R}_{g} cos ( - {ζ}_{1} + \frac{π}{6} ) {θ}_{1} = {l}_{1 b 1} - {l}_{1 a 1} & ( 45 )\end{matrix}$$

In the same way, the distance R_{ca} between the wire 1a and wire 1c on the W₁-Z₁ plane can be expressed by the following Expression (46). [Math. 14]

$$\begin{matrix}[ Math . 14 ] & \\ {R}_{ca} = \frac{\sqrt{3}}{2} {R}_{g} cos ( {ζ}_{1} + \frac{π}{6} ) & ( 46 )\end{matrix}$$

Accordingly, the following Expression (47) holds between the bending angle θ₁ and
lengths l₁ₐ₁ and l_{1c1}.

$$\begin{matrix}[ Math . 15 ] & \\ \frac{\sqrt{3}}{2} {R}_{g} cos ( {ζ}_{1} + \frac{π}{6} ) {θ}_{1} = {l}_{1 c 1} - {l}_{1 a 1} & ( 47 )\end{matrix}$$

From Expressions (45) and (47), the driving amount l_{k1b} of the wire 1b and the driving amount l_{k1c} of the wire 1c when assuming the wires to be rigid can be expressed by the following Expressions (48) and (49).

$$\begin{matrix}[ Math . 16 ] & \\ {I}_{k 1 b} = \frac{\sqrt{3}}{2} {R}_{g} cos ( - {ζ}_{1} + \frac{π}{6} ) {θ}_{1} & ( 48 )\end{matrix}$$

$${I}_{k 1 c} = \frac{\sqrt{3}}{2} {R}_{g} cos ( {ζ}_{1} + \frac{π}{6} ) {θ}_{1} \qquad ( 49 )$$

Next, an elastic wire driving model is derived taking into consideration stretching and contraction of the wires. The lengths l₁ₐ₁ and l_{1b1} in the continuum robot 310 according to the present embodiment, and the drive control amount l_{p1b} of the wire 1b are expressed by Expressions (3) through (5), in the same way as in the first embodiment. The wire length l_{1c1} can be expressed by the following Expression (50) using the drive control amount l_{p1c} of the wire 1c, in the same way as with the wire length l_{1b1}.

$${1}_{1 c 1} = {1}_{10} + {Δ1}_{1 c} + {1}_{p 1 c} \qquad ( 50 )$$

Accordingly, the drive control amount l_{p1c} of the wire 1c is expressed by the following Expression (51).

$${1}_{p 1 c} = {1}_{k 1 c} + {Δ1}_{1 a} - {Δ1}_{1 c} \qquad ( 51 )$$

It can be seen from this Expression (51) that the driving amount can be computed taking into consideration stretching and contraction of the wires in the continuum robot 310 according to the present embodiment, by adding the driving amount derived from the kinematic model to difference in wire displacements. Accordingly, a compensation amount l_{e1c} of the wire 1c can be expressed by the following Expression (52).

$${1}_{e 1 c} = {Δ1}_{1 a} - {Δ1}_{1 c} \qquad ( 52 )$$

An expression of balance of the force and moment acting on the distal end 3126 of the bendable portion 312 is derived to obtain the displacement of the wires. The balance of force can be expressed by the following Expression (53) using the forces f₁ₐ, f_{1b}, and f_{1c}, since the distal end 3126 of the bendable portion 312 is under force in the Z₂ axis direction from the wire 1a, wire 1b, and wire 1c.

$${f}_{1 a} + {f}_{1 b} + {f}_{1 c} = 0 \qquad ( 53 )$$

Bending moments M₁ₐ₁, M_{1b1}, and M_{1c1} on the Y₂ axis from the wire 1a, wire 1b, and wire 1c, act on the distal end 3126 of the bendable portion 312, and bending moment is balanced with the moment generated by the forces f₁ₐ, f_{1b}, and f_{1c} received from the wires. Accordingly, the balance of moments on the Y₂ axis can be expressed by the following Expression (54).

$$\begin{matrix}[ Math . 17 ] & \\ \frac{{R}_{g}}{2} \{ {f}_{1 a} sin ( - {ζ}_{1} - \frac{π}{2} ) + {f}_{1 b} sin ( - {ζ}_{1} + \frac{π}{6} ) + {f}_{1 c} sin ( - {ζ}_{1} + \frac{5 π}{6} ) \} - {M}_{1 a 1} - {M}_{1 b 1} - {M}_{1 c 1} = 0 & ( 54 )\end{matrix}$$

Moment due to the forces f₁ₐ, f_{1b}, and f_{1c} is generated on the X₂ axis, so the balance Expression can be expressed by the following Expression (55).

$$\begin{matrix}[ Math . 18 ] & \\ \frac{{R}_{g}}{2} \{ {f}_{1 a} sin ( - {ζ}_{1} ) + {f}_{1 b} sin ( - {ζ}_{1} + \frac{2 π}{3} ) + {f}_{1 c} sin ( - {ζ}_{1} + \frac{4 π}{3} ) \} = 0 & ( 55 )\end{matrix}$$

Note that the force and moment of the wire 1c can be expressed by the following Expressions (56) and (57) respectively, in the same way as with the wire 1a and the wire 1b.

$$\begin{matrix}[ Math \text{.19} ] & \\ {f}_{1 c} = - {k}_{e} Δ {l}_{1 c} & ( 56 )\end{matrix}$$

$${M}_{1 c 1} = - \frac{{k}_{m}}{{l}_{1 c 1}} {θ}_{1} \qquad ( 57 )$$

### (3-2. Control System)

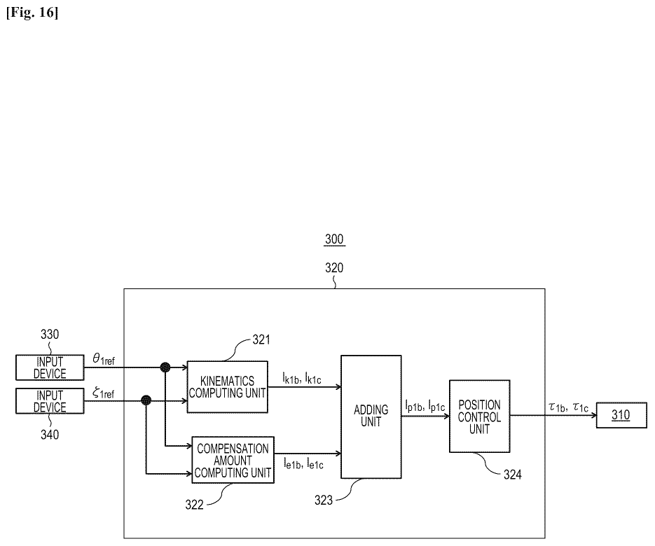

In the third embodiment, numerical solutions of the displacements Δl₁ₐ, Δl_{1b}, and Δl_{1c} of the wires are obtained using iterative calculation, in the same way as with the abovedescribed second embodiment. FIG. 16 is a diagram illustrating an example of the functional configuration of a continuum robot control system 300 according to the third embodiment of the present invention. The continuum robot control system 300 illustrated in FIG. 16 is configured including the continuum robot 310 illustrated in FIG. 14, a continuum robot control device 320, an input device 330, and an input device 340.

The continuum robot control device 320 sets an drive control amount l_{p1b} for diving control of the wire 1b and an drive control amount l_{p1c} for diving control of the wire 1c, based on the target bending angle θ_{1ref} of the bendable portion 312 input from the input device 330, and target rotation angle ζ_{1ref} of the bendable portion 312 input from the input device 340, and outputs a driving command τ_{1b} based on the drive control amount l_{p1b} and a driving command τ_{1c} based on the drive control amount l_{p1c} to the continuum robot 310 (or more specifically, the actuators 311b and 311c). That is to say, the third embodiment differs from the above-described first embodiment with regard to the point that, in addition to input of the target bending angle θ_{1ref} from the input device 330, input of the target rotation angle ζ_{1ref} from the input device 340 is also performed.

The continuum robot control device 320 illustrated in FIG. 16 is configured including a kinematics computing unit 321, a compensation amount computing unit 322, an adding unit 323, and a position control unit 324.

The kinematics computing unit 321 calculates the driving amount l_{k1b} and l_{k1c} for each of the wires 1b and 1c, based on the target bending angle θ_{1ref} of the bendable portion 312 input from the input device 330, the target rotation angle ζ_{1ref} of the bendable portion 312 input from the input device 340, and the distance between the wires 1b and 1c (predetermined wires) and wire 1a (other wire). Specifically, the kinematics computing unit 321 calculates the driving amount l_{k1b} and l_{k1c} based on the kinematics model, using Expressions (48) and (49).

The compensation amount computing unit 322 computes the compensation amounts l_{e1b} and l_{e1c} for each of the wires 1b and 1c, based on the target bending angle θ_{1ref} of the bendable portion 312 input from the input device 330, the target rotation angle ζ_{1ref} of the bendable portion 312 input from the input device 340, and the displacements Δl₁ₐ, Δl_{1b}, and Δl_{1c} of the wires. Specifically, the compensation amount computing unit 322 first obtains the displacements Δl₁ₐ, Δl_{1b}, and Δl_{1c} of the wires by iterative calculation, and then calculates the compensation amounts l_{e1b} and l_{e1c}, which are the differences of the displacements.

The adding unit 323 adds the driving amounts l_{k1b} and l_{k1c} calculated by the kinematics computing unit 321 and the compensation amounts 1_{e1b} and l_{e1c} calculated by the compensation amount computing unit 322, for each of the wires 1b and 1c, thereby computing the drive control amounts l_{p1b} and l_{p1c}. That is to say, the adding unit 323 performs processing of compensating the driving amounts l_{k1b} and l_{k1c} calculated by the kinematics computing unit 321 by the compensation amounts l_{e1b} and l_{e1c} calculated by the compensation amount computing unit 322, to calculate the drive control amount l_{p1b} of the wire 1b and the drive control amount l_{p1c} of the wire 1c. The position control unit 324 outputs a drive command 11 based on the drive control amount l_{p1b} and a drive command τ_{1b} based on the drive control amount l_{p1c} to the continuum robot 310 (or more specifically, to the actuators 311b and 311c). In the present embodiment, the adding unit 323 and position control unit 324 make up a setting device that sets the drive control amount l_{p1b} of the wire 1b and the drive control amount l_{p1c} of the wire 1c.

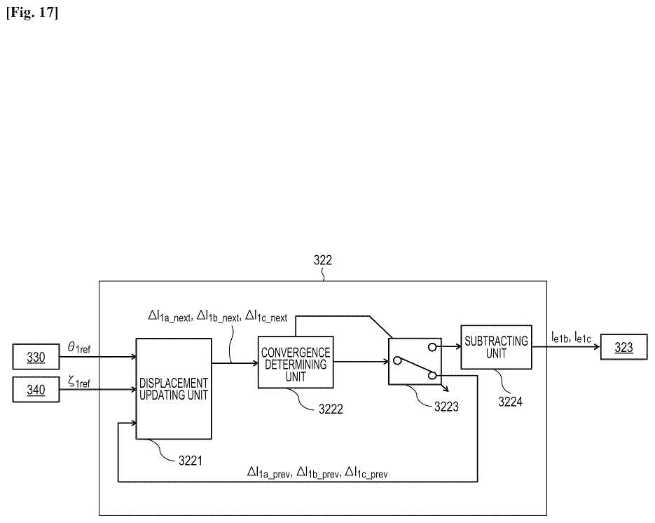

FIG. 17 is a diagram illustrating an example of the functional configuration of the compensation amount computing unit 322 illustrated in FIG. 16, according to the third embodiment of the present invention. The compensation amount computing unit 322 is configured including a displacement updating unit 3221, a convergence determining unit 3222, an output switching unit 3223, and a subtracting unit 3224, as illustrated in FIG. 17.

In accordance with input of the target bending angle θ_{1ref} and target rotation angle ζ_{1ref}, and first displacement candidate values Δl_{1a_prev}, Δl_{1b_prev}, and Δl_{1c_prev} of the displacements of the wires 1a, 1b, and 1c, the displacement updating unit 3221 updates to second displacement candidate values Δl_{1a_next}, Δl_{1b_next}, and Δl_{1c_next}. At this time, the displacement updating unit 3221 updates the second displacement candidate values Δl_{1a_next}, Δl_{1b_next}, and Δl_{1c_next} of the displacement of the wires 1a, 1b, and 1c, using the elastic wire driving model described above.

The convergence determining unit 3222 determines whether or not the second dis-placement candidate values Δl₁ₐ next, Δl_{1b_next}, and Δl_{1c_next} have converged at a constant value. In a case of having determined that the second displacement candidate values Δl_{1a_next}, Δl_{1b_next}, and Δl_{1c_next} have converged at a constant value, the convergence determining unit 3222 causes the output switching unit 3223 to output the second displacement candidate values Δl_{1a_next}, Δl_{1b_next}, and Δl_{1c_next} as displacements Δl₁ₐ, Δl_{1b}, and Δl_{1c}, to the subtracting unit 3224. In a case of having determined that the second displacement candidate values Δl₁ₐ next, Δl_{1b_next}, and Δl_{1c_next} have not converged at a constant value, the convergence determining unit 3222 causes the output switching unit 3223 to output the second displacement candidate values Δl_{1a_next}, Δl_{1b_next}, and Δl_{1c_next} as first displacement candidate values Δl_{1a_prev}, Δl_{1b_prev}, and Δl_{1c_prev} to the displacement updating unit 3221, and iterative calculation is performed again. The convergence determining unit 3222 and output switching unit 3223 according to the present embodiment make up a configuration corresponding to an output processing unit according to the present invention.

The subtracting unit 3224 is a compensation amount calculating unit that uses the second displacement candidate values Δl_{1a_next}, Δl_{1b_next}, and Δl_{1c_next} as displacements Δl₁ₐ, Δl_{1b}, and Δl_{1c}, respectively, to calculate the compensation amounts l_{e1b} and l_{e1c}, respectively, from Expression (52) and so forth.

Note that an arrangement may be made where, in a case that the second displacement candidate values Δl₁ₐ next, Δl_{1b_next}, and Δl_{1c_next} do not converge at a constant value within a predetermined iteration count, the convergence determining unit 3222 causes the output switching unit 3223 to output the last second displacement candidate values Δl_{1a_next}, Δl_{1b_next}, and Δl_{1c_next} to have converged, as displacements Δl₁ₐ, Δl_{1b}, and Δl_{1c}, respectively, to the subtracting unit 3224. This can prevent the compensation amounts calculated at the subtracting unit 3224 from changing discontinuously. Although the initial values of the first displacement candidate values Δl_{1a_prev}, Δl_{1b_prev}, and Δl_{1c_prev} in the iterative calculation according to the present embodiment are 0, the displacements that converged last may be used as the initial values.

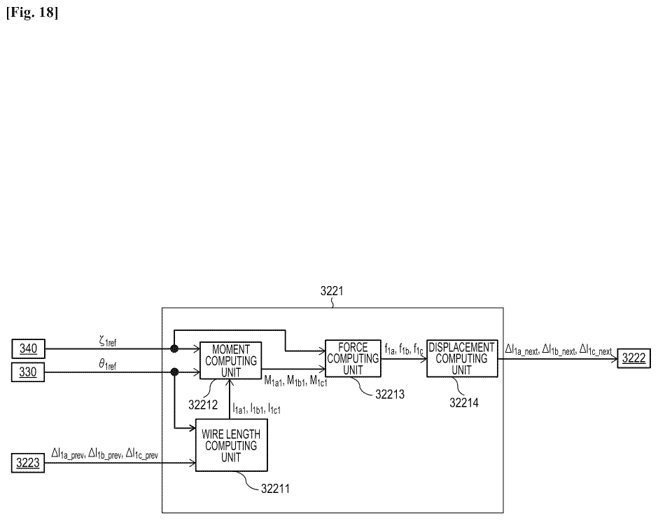

FIG. 18 is a diagram illustrating an example of the functional configuration of the displacement updating unit 3221 illustrated in FIG. 17, according to the third embodiment of the present invention. The displacement updating unit 3221 is configured including a wire length computing unit 32211, a moment computing unit 32212, a force computing unit 32213, and a displacement computing unit 32214, as illustrated in FIG. 18.

The wire length computing unit 32211 calculates the wire lengths l₁ₐ₁, l_{1b1}, and l_{1c1}, for each of the multiple wires 1a, 1b, and 1c at the bendable portion 312, in accordance with the input of the target bending angle θ_{1ref} and the first displacement candidate values Δl_{1a_prev}, Δl_{1b_prev} and Δl_{1c_prev}. Specifically, the wire length computing unit 32211 calculates the wire lengths l₁ₐ₁, l_{1b1}, and l_{1c1}, using the following Expressions (58) through (60), where Expressions (3), (5), and (48) through (51) have been modified.

$$\begin{matrix}[ Math \text{.20} ] & \\ {l}_{1 a 1} = {l}_{10} + Δ {l}_{1 a \_ prev} & ( 58 )\end{matrix}$$

$${l}_{1 b 1} = {l}_{10} + Δ {l}_{1 a \_ prev} + \frac{\sqrt{3}}{2} {R}_{g} cos ( - {ζ}_{1} + \frac{π}{6} ) {θ}_{1 ref} \qquad ( 59 )$$

$${l}_{1 c 1} = {l}_{10} + Δ {l}_{1 a \_ prev} + \frac{\sqrt{3}}{2} {R}_{g} cos ( {ζ}_{1} + \frac{π}{6} ) {θ}_{1 ref} \qquad ( 60 )$$

The moment computing unit 32212 calculates bending moments M₁ₐ₁, M_{1b1}, and M_{1c1}, for each of the multiple wires 1a, 1b, and 1c, in accordance with the target bending angle θ_{1ref}, target rotation angle ζ_{1ref}, and the wire lengths l₁ₐ₁, l_{1b1}, and l_{1c1} of the wires. Specifically, the moment computing unit 32212 calculates the bending moments M₁ₐ₁, M_{1b1}, and M_{1c1}, using Expressions (10) and (57).

The force computing unit 32213 calculates forces f₁ₐ, f_{1b}, and f_{1c} representing the tensile force on the wires 1a, 1b, and 1c, in accordance with the input of the bending moments M₁ₐ₁, M_{1b1}, and M_{1c1}, of the multiple wires 1a, 1b, and 1c. Specifically, the force computing unit 32213 calculates the forces f₁ₐ, f_{1b}, and f_{1c} using Expressions (53) through (55).

The displacement computing unit 32214 computes the second displacement candidate values Δl_{1a_next}, Δl_{1b_next}, and Δl_{1c_next} in accordance with the forces f₁ₐ, f_{1b}, and f_{1c} of the multiple wires 1a, 1b, and 1c, and updates displacement candidate values. Specifically, the displacement computing unit 32214 uses Expressions (9) and (56) to compute the second displacement candidate values Δl_{1a_next}, Δl_{1b_next}, and Δl_{1c_next}, and updates the displacement candidate values.

### (3-3. Simulation)

The control system according to the third embodiment, illustrated in FIGS. 16 through 18, is applied to the continuum robot 310 having the bendable portion 312 that is threedimensionally bendable. Note that the length of the bendable portion 312 is 0.01 m, and the length of the elongated portion 111 is 1 m.

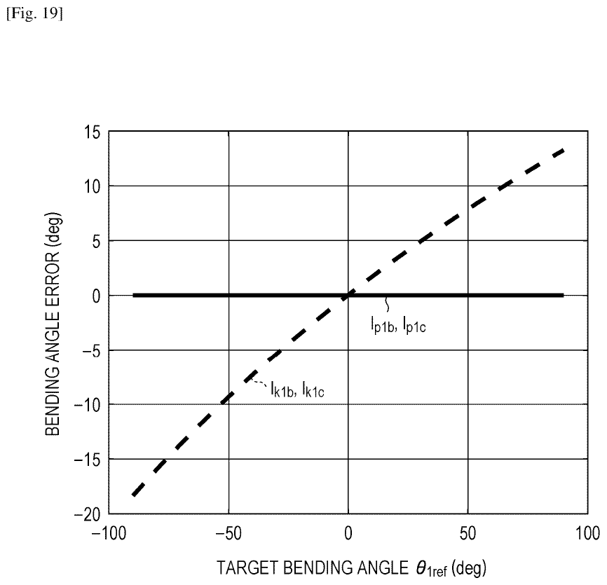

FIG. 19 is a characteristics diagram illustrating the relation between target bending angle and bending angle error. The horizontal axis in the characteristics diagram of FIG. 19 is the target bending angle θ_{1ref}, and the vertical axis is the bending angle error.

Specifically, FIG. 19 shows bending angle error of the bendable portion 312 in a case where the target rotation angle ζ_{1ref} is 0 degrees, and the target bending angle θ_{1ref} is changed from −90 degrees to 90 degrees. The dashed line in FIG. 19 illustrates a comparative example of a case where the driving amounts l_{k1b} and l_{k1c} calculated using the kinematics model are used as control input. It can be understood that in this case, the angle error of the bendable portion 312 increases in accordance with the increase or decrease of the target bending angle θ_{1ref}. On the other hand, the solid line illustrates the present embodiment in a case of using the drive control amounts l_{p1b} and l_{p1c} taking stretching and contraction of the wires into consideration. It can be understood that in this case, the angle error of the bendable portion 312 is 0 even if there is change in the target bending angle θ_{1ref}.

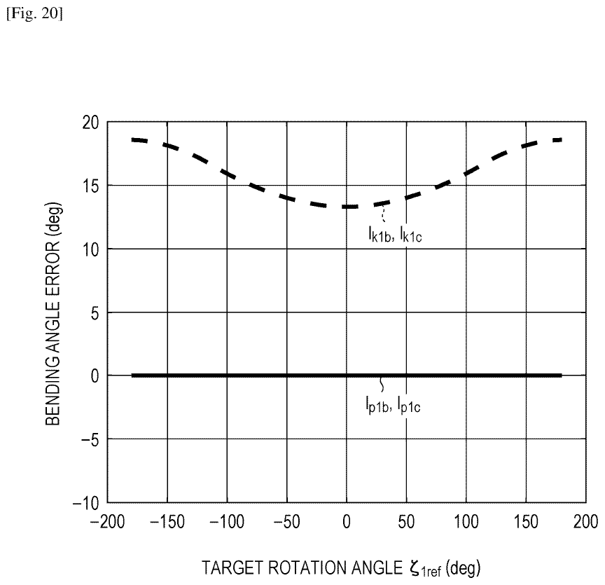

FIG. 20 is a characteristics diagram illustrating the relation between target rotation angle and bending angle error according to the third embodiment of the present invention. The horizontal axis in the characteristics diagram of FIG. 20 is the target rotation angle ζ_{1ref}, and the vertical axis is the bending angle error.

Specifically, FIG. 20 shows bending angle error of the bendable portion 312 in a case where the target bending angle θ_{1ref} is 90 degrees, and the target rotation angle ζ_{1ref} is changed from-180 degrees to 180 degrees. The dashed line in FIG. 20 illustrates a comparative example of a case where the driving amounts l_{k1b} and l_{k1c} calculated using the kinematics model are used as control input. It can be understood that in this case, angle error of the bendable portion 312 occurs dependent on the target rotation angle ζ_{1ref}. On the other hand, the solid line illustrates the present embodiment in a case of using the drive control amounts l_{p1b} and l_{p1c} taking stretching and contraction of the wires into consideration as control input. It can be understood that in this case, the angle error of the bendable portion 312 is 0 regardless of the target rotation angle ζ_{1ref}. From the above, it can be understood that attitude error of the continuum robot 310 having the bendable portion 312 that can be three-dimensionally bent can be reduced, by applying the control system illustrated in FIGS. 16 through 18 according to the present embodiment.

In the continuum robot control device 320 according to the third embodiment, the compensation amount computing unit 322 computes the respective compensation amounts l_{e1b} and l_{e1c} for the wires 1b and 1c, based on the target bending angle θ_{1ref} of the bendable portion 312 input from the input device 330, and the target rotation angle ζ_{1ref} of the bendable portion 312 input from the input device 340, for each wire 1b and 1c (predetermined wires) of the bendable portion 312. The adding unit 323 and position control unit 324 perform processing to compensate the respective driving amounts l_{k1b} and l_{k1c} calculated at the kinematics computing unit 321, by the compensation amounts l_{e1b} and l_{e1c} calculated at the compensation amount computing unit 322, thereby setting the drive control amounts l_{p1b} and l_{p1c} of the wires 1b and 1c. According to this configuration, error in the driving amounts l_{k1b} and l_{k1c} due to wire deformation can be compensated for in the continuum robot 310 having the bendable portion 312 that is three-dimensionally bendable, as well. Accordingly, error between the reference position of the bendable portion 312 and the actual attitude can be reduced, and as a result, reduction in the risk of contact between a path in the body or the like and the bendable portion can be realized.

The driving amount of each wire can be compensated for in the present embodiment using the compensation amount calculated based on the displacement of any one wire of the multiple wires relating to the same bendable portion, in the same way as in the first embodiment.

### Fourth Embodiment

Next, a fourth embodiment of the present invention will be described. The fourth embodiment is an arrangement of applying a continuum robot having multiple bendable portions that are bendable three-dimensionally, by combining the control system according to the second embodiment and the control system according to the third embodiment. Specifically, the control system according to the fourth embodiment computes displacement of the wires that drive the bendable portions, using iterative calculation. When carrying out iterative calculation, the elastic wire driving model that takes into consideration coupling of force and moment acting among the bendable portions is used in the same way as in the second embodiment. Further, the control system according to the fourth embodiment obtains the driving amount from the target bending angle and target rotation angle.

### (4-1. Modelling)

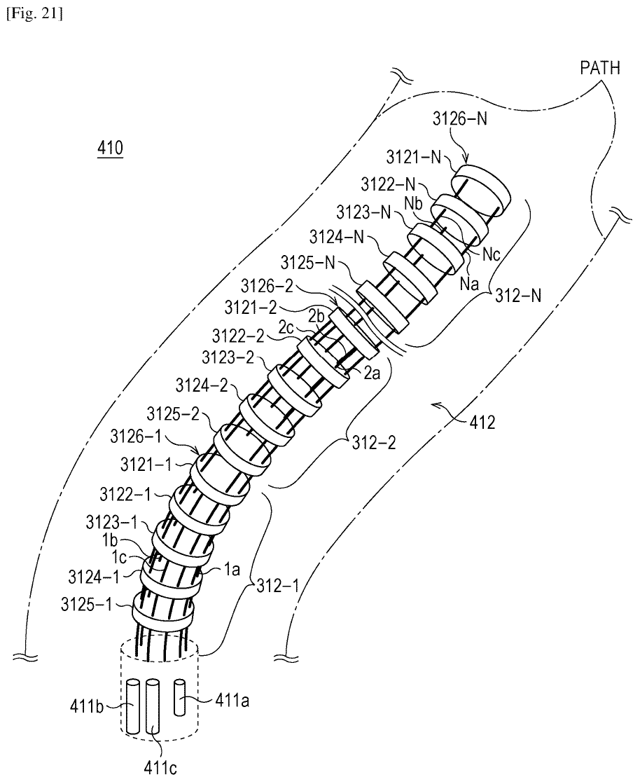

FIG. 21 is a diagram illustrating an example of a schematic configuration of a continuum robot 410 used in the fourth embodiment of the present invention. Configurations that are the same as configurations illustrated in FIGS. 1, 2, 7, and 14 are denoted by the same symbols, and detailed description thereof will be omitted. FIG. 21 also illustrates an example of a path into which a bendable portions 412 is inserted and removed from.

The bendable portion 412 is configured having multiple (specifically, N) bendable portions 312 illustrated in FIG. 14 in the direction of inserting to and removing from the path. That is to say, this continuum robot 410 illustrated in FIG. 21 is configured having multiple bendable portions 312-1 through 312-N, arranged serially. One end of a wire an, wire nb, and wire nc, is connected to a distal end 3126-n of an n'th bendable portion (where n=1, 2, . . . , N) of the bendable portion 412. The wires na, nb, and nc are guided from an n−1′th bendable portion 312-(n−1) to wire guides of a first bendable portion 312-1 and the elongated portion 111. The wire na is connected to a fixing portion 411a, the wire nb is connected to an actuator 411b, and the wire nc is connected to an actuator 411c. Note that in the example illustrated in FIG. 21, the actuator 411b is a driving mechanism that can drive each of wires 1b through Nb independently. Also note that an arrangement may be made where N actuators 411b corresponding to the wires 1b through Nb are configured. In the same way, the actuator 411c is a driving mechanism that can drive each of wires 1c through Nc independently, and an arrangement may be made where N actuators 411c corresponding to the wires 1c through Nc are configured. In the present embodiment, the bending angle θₙ and rotation angle ζₙ at the n'th bendable portion 312-n are controlled by driving control of the actuators 411b and 411c.

### (4-2. Control System)

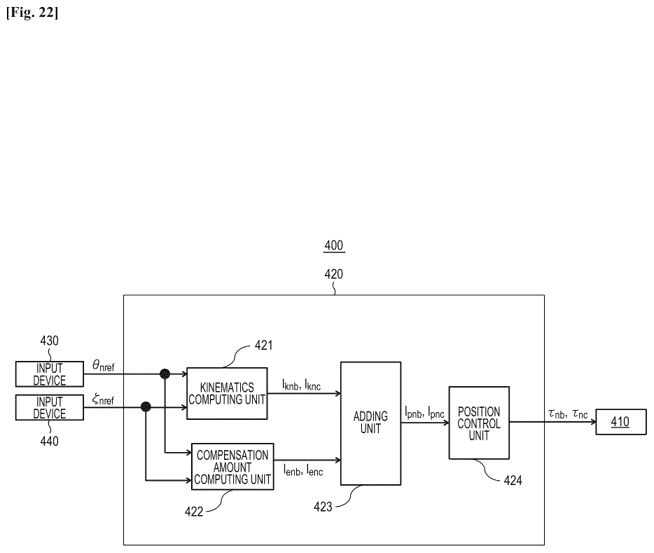

FIG. 22 is a diagram illustrating an example of the functional configuration of a continuum robot control system 400 according to the fourth embodiment of the present invention. The continuum robot control system 400 illustrated in FIG. 22 is configured including the continuum robot 410 illustrated in FIG. 21, a continuum robot control device 420, an input device 430, and an input device 440.

The kinematics computing unit 421 accepts target bending angle θ_{nref} and target rotation angle ζ_{nref} (where n=1, 2, . . . , N) from the input device 430 and input device 440 respectively, for the first bendable portion 312-1 through the N'th bendable portion 312-N, and calculates the driving amount l_{knb} and l_{knc} for each bendable portion using the kinematic model in the same way as the third embodiment.

The compensation amount computing unit 422 accepts input of target bending angle θ_{nref} and target rotation angle ζ_{nref} (where n=1, 2, . . . , N) from the input device 430 and input device 440 respectively, for the first bendable portion 312-1 through the N'th bendable portion 312-N, and calculates the compensation amounts l_{enb} and l_{enc} of the bendable portions. At this time, the compensation amount computing unit 422 calculates the displacements Δlₙₐ, Δl_{nb}, and Δl_{nc} using the elastic wire driving model that takes into consideration coupling of force and moment acting among the bendable portions, and iterative calculation, in the same way as in the second embodiment, and calculates the compensation amounts l_{enb} and l_{enc} from these displacements.

The adding unit 423 adds the driving amounts l_{knb} and 1_{knc} calculated by the kinematics computing unit 421 and the compensation amounts 1_{e1b} and l_{enc} calculated by the compensation amount computing unit 422, thereby computing the drive control amounts l_{pnb} and l_{pnc}. That is to say, the adding unit 423 performs processing of compensating the driving amounts l_{knb} and l_{knc} calculated by the kinematics computing unit 421 respectively by the compensation amounts l_{cnb} and l_{enc} calculated by the compensation amount computing unit 422, to calculate the drive control amount l_{pnb} of the wire nb and the drive control amount l_{pnc} of the wire nc. The position control unit 424 outputs a drive command τ_{nb} based on the drive control amount l_{pnb} and a drive command τ_{nc} based on the drive control amount l_{pnc} to the continuum robot 410 (or more specifically, to the actuators 411b and 411c). In the present embodiment, the adding unit 423 and position control unit 424 make up a setting device that sets the drive control amount l_{pnb} for driving control of the wire nb and the drive control amount l_{pnc} for driving control of the wire nc.

According to the fourth embodiment, error in the driving amounts l_{knb} and l_{knc} due to wire deformation can be compensated for in the continuum robot 410 having the multiple bendable portions 412 that are three-dimensionally bendable, as well. Accordingly, error between the target posture of the bendable portion 412 and the actual attitude can be reduced, and as a result, reduction in the risk of contact between a path in the body or the like and the bendable portion can be realized.

The driving amount of each wire can be compensated for in the present embodiment using the compensation amount calculated based on the displacement of any one wire of the multiple wires relating to the same bendable portion, in the same way as in the first embodiment.

### Fifth Embodiment

Next, a fifth embodiment of the present invention will be described. The abovedescribed first through fourth embodiments have been arrangements presenting a control system that obtains driving amount control, taking into consideration stretching and contraction of the wires, when a certain reference angle is provided. The fifth embodiment is an arrangement where, when a reference path in which a reference angle (e.g., target bending angle) changes in a manner dependent on the amount of movement of the distal end of a bendable portion in the longitudinal direction, the attitude of the bendable portion is automatically controlled to follow the path.

The continuum robots according to the above-described first through fourth embodiments are dependent on the amount of wire driving for the position of the distal end of the bendable portion, so the reference angle when following a reference path will be dependent on the amount of wire driving. At this time, the amount of wire driving cannot be directly obtained using the control systems in the above-described first through fourth embodiments. The reason is that the control systems in the abovedescribed first through fourth embodiments need a reference angle to be set, to obtain the driving amount. Accordingly, the control system according to the fifth embodiment obtains the driving control amount by performing iterative calculation, where updating is repeated until the reference angle and amount of wire driving converge to a constant value. Specifically, in the fifth embodiment, a candidate value for reference angle is first input, and a candidate value for the amount of wire driving is computed using the control system according to the first embodiment. Next, the distal end position corresponding to the candidate value for the driving amount is computed, and the candidate value for the reference angle is updated based on the distal end position and reference path. This computation is repeated until the candidate value for the driving amount converges.

### (5-1. Modelling)

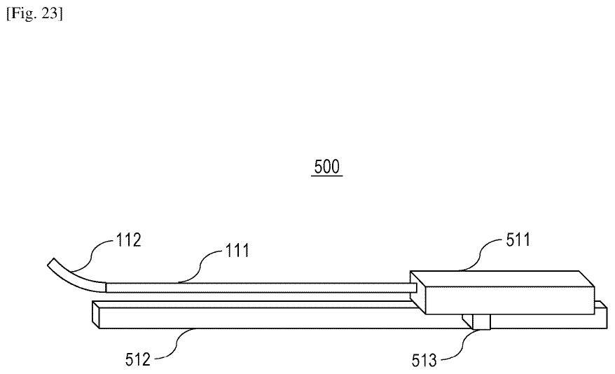

FIG. 23 is a diagram illustrating an example of the external configuration of a continuum robot control system 500 used in the fifth embodiment of the present invention. The continuum robot control system 500 illustrated in FIG. 23 is configured including the elongated portion 111 and bendable portion 112 illustrated in FIG. 1, and a base portion 511, a linear guide 512, and a position detector 513.

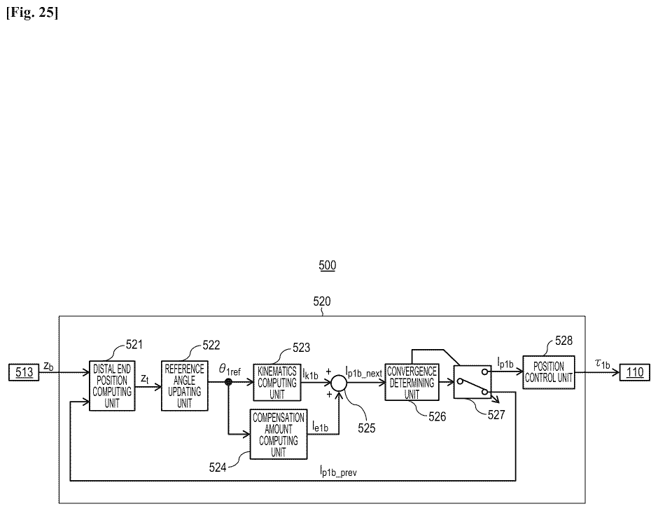

The continuum robot according to the fifth embodiment is the continuum robot 110 illustrated in FIG. 1, configured having one bendable portion 112, in the same way as the first embodiment. The base portion 511 is fixed to the linear guide 512, and the continuum robot 110 in the fifth embodiment advances and retracts along the linear guide 512 by pushing and pulling the base portion 511. The position detector 513 that measures movement amount z_{b} of the linear guide 512 is attached to the linear guide 512. This movement amount z_{b} of the linear guide 512 is input from the position detector 513 to a continuum robot control device 520 illustrated in FIG. 25, which will be described later. Before driving the linear guide 512, a target bending angle path Θ (zₜ) is input from a higher-order device (omitted from illustration) to the continuum robot control device 520 illustrated in FIG. 25, which will be described later. The target bending angle path Θ (zₜ) in the present embodiment is a function of a movement amount zₜ of the distal end 1126 of the bendable portion 112.

### (5-2. Control System)

The control system according to the fifth embodiment finds a drive control amount l_{p1b} where the bending angle θ₁ of the distal end 1126 matches the target bending angle path Θ (zₜ). At this time, the drive control amount l_{p1b} is a function of the target bending angle path Θ (zₜ), so the drive control amount l_{p1b} cannot be obtained with the bending angle θ_{1ref} as a constant, which has been described in the first embodiment. Accordingly, the drive control amount l_{p1b} is obtained by iterative calculation in the fifth embodiment. First, a method for computing the movement amount zₜ of the distal end 1126 of the bendable portion 112 will be described.

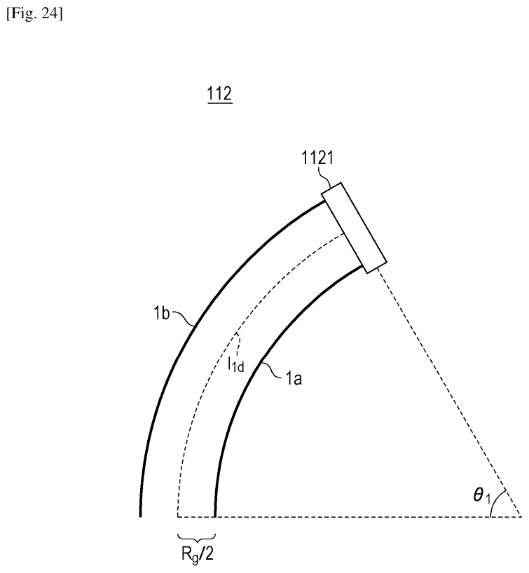

FIG. 24 is a diagram illustrating an example of kinematic model of the bendable portion 112 illustrated in FIG. 23, according to the fifth embodiment of the present invention. With the length of an axis connecting the distal end 1126 of the bendable portion 112 and the center of each wire guide as l_{1d}, this l_{1d} can be expressed by the following Expression (61) using the drive control amount l_{p1b}.

$$\begin{matrix}[ Math \text{.21} ] & \\ {l}_{1 d} = {l}_{10} + \frac{{l}_{p 1 b}}{2} & ( 61 )\end{matrix}$$

Accordingly, the movement amount zₜ of the distal end 1126 of the bendable portion 112 can be expressed by the following Expression (62) using the movement amount z_{b} of the linear guide 512.

$$\begin{matrix}[ Math \text{.22} ] & \\ {z}_{t} = {l}_{0} + \frac{{l}_{p 1 b}}{2} + {z}_{b} & ( 62 )\end{matrix}$$

Next, the functional configuration of the continuum robot control system 500 according to the fifth embodiment will be described. FIG. 25 is a diagram illustrating an example of the functional configuration of the continuum robot control system 500 according to the fifth embodiment of the present invention. The continuum robot control system 500 illustrated in FIG. 25 is configured including the continuum robot 110 illustrated in FIG. 1, the continuum robot control device 520, and the position detector 513.

The continuum robot control device 520 is configured including a distal end position computing unit 521, a reference angle updating unit 522, a kinematics computing unit 523, a compensation amount computing unit 524, an adding unit 525, a convergence determining unit 526, an output switching unit 527, and a position control unit 528.

The distal end position computing unit 521 is a third computing device that computes the movement amount zₜ of the distal end 1126 of the bendable portion 112 in accordance with the movement amount (movement amount of the continuum robot 110 in the longitudinal direction) z_{b} of the linear guide 512, and input of a first driving control amount candidate value l_{p1b_prev} in the driving control amount of the wire 1b (predetermined wire). Specifically, the distal end position computing unit 521 calculates the movement amount zₜ of the distal end 1126 of the bendable portion 112 using Expression (62).

The reference angle updating unit 522 updates the target bending angle θ_{1ref} corresponding to the movement amount zₜ, in accordance with the movement amount zₜ of the distal end 1126 from the distal end position computing unit 521 and input of the target bending angle path Θ (zₜ) from the higher-order device (omitted from illustration) described above.

The kinematics computing unit 523 calculates the driving amount l_{k1b} of the wire 1b, in accordance with input of the target bending angle θ_{1ref} from the reference angle updating unit 522, in the same way as in the first embodiment. Further, the compensation amount computing unit 524 calculates the compensation amount l_{e1b} of the wire 1b, in accordance with input of the target bending angle θ_{1ref} from the reference angle updating unit 522, in the same way as in the first embodiment.

The adding unit 525 adds the driving amount l_{k1b} calculated at the kinematics computing unit 523 and the compensation amount l_{e1b} calculated at the compensation amount computing unit 524, and calculates a second driving control amount candidate value l_{p1b_next} for the driving control amount of the wire 1b. The convergence determining unit 526 determines whether or not the second driving control amount candidate value l_{p1b_next} has converged to a constant value. In a case of having determined that the second driving control amount candidate value l_{p1b_next} has converged to a constant value, the convergence determining unit 526 causes the output switching unit 527 to output the second driving control amount candidate value l_{p1b_next} to the position control unit 528 as the drive control amount l_{p1b}. In a case of having determined that the second driving control amount candidate value l_{p1b_next} has not converged to a constant value, the convergence determining unit 526 causes the output switching unit 527 to output the second driving control amount candidate value l_{p1b_next} to the distal end position computing unit 521 as the first driving control amount candidate value l_{p1b_prev}, and iterative calculation is performed again. The position control unit 528 outputs a driving command th based on the drive control amount l_{p1b}, to the continuum robot 110 (or more specifically, the actuator 113), in the same way as in the first embodiment. In the present embodiment, the adding unit 525, convergence determining unit 526, output switching unit 527, and position control unit 528 make up a setting device that sets the drive control amount l_{p1b} for diving control of the wire 1b.

Note that while a control system of performing control with regard to the continuum robot 110 having one bendable portion 112 has been described in the present embodiment, this is similarly applicable to the continuum robot 410 having multiple bendable portions 412 that are three-dimensionally bendable, for example.

### Sixth Embodiment

Next, a sixth embodiment of the present invention will be described. Description of items that are the same as in the above-described first through fifth embodiments will be omitted from the following description of the sixth embodiment, and description will be made regarding items that are different from the above-described first through fifth embodiments.

The sixth embodiment is an arrangement where a continuum robot of which the rigidity of the bendable portion changes by a jacket or by insertion of surgical instruments is applied. Specifically, the continuum robot according to the sixth embodiment is suitable for use as a flexible endoscope described in the Background Art section. This type of continuum robot preferably has the surface thereof covered by a smooth jacket so that body tissue is not damaged even if the continuum robot comes into contact with the tissue, for example. There is need to perform bending driving of the jacket along with the bendable portion, so there is need to drive the wires with a larger tensile force as compared to a continuum robot that has no jacket. Some flexible endoscopes have a hollow structure for insertion of surgical instruments such as forceps of the like, used for bioptic sampling of diseased tissue, or the like. In order to drive the bendable portion of the continuum robot in a state where a surgical instrument is inserted, the surgical instrument has to be bent as well, so there is need to drive the wires with a larger tensile force as compared to when no surgical instrument is inserted. Accordingly, the rigidity of the bendable portion differs depending on the presence or absence of jacket and surgical instrument. In light of this point, the control systems in the above-described first through fifth embodiments only take into consideration the rigidity of the wires when obtaining the displacement of the wires, so angle error cannot be accurately compensated for if the rigidity of the bendable portion changes depending on the presence or absence of jacket and surgical instrument. Accordingly, the sixth embodiment illustrates an example of a control system where the rigidity of the bendable portion can be specified as a variable.

### (6-1. Modelling)

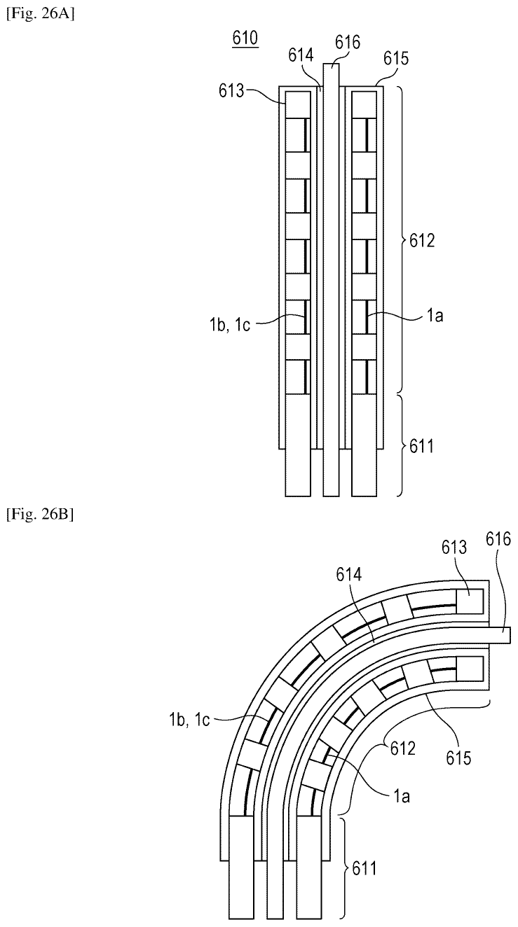

FIGS. 26A and 26B are diagrams illustrating an example of a schematic configuration of a continuum robot 610 used in the sixth embodiment of the present invention. More specifically, FIGS. 26A and 26B are diagrams illustrating cross-sections of the continuum robot 610 used in the sixth embodiment of the present invention, taken along a plane parallel to the longitudinal direction of a bendable portion 612. Note that the continuum robot 610 according to the present embodiment is capable of bending three-dimensionally, in the same way as the continuum robot 310 in the third embodiment illustrated in FIG. 14. Note that the continuum robot 610 illustrated in FIGS. 26A and 26B has the multiple wires 1a, 1b, and 1c, and also is configured including the fixing portion 311a to which the wire 1a is connected, the actuator 311b to which the wire 1b is connected, and the actuator 311c to which the wire 1c is connected, which are illustrated in FIG. 14, although omitted from illustration in FIGS. 26A and 26B, in the same way as the continuum robot 310 according to the third embodiment illustrated in FIG. 14.

The continuum robot 610 according to the present embodiment is configured including an elongated portion 611 and a bendable portion 612, as illustrated in FIG. 26A. Multiple wire guides 613 are also provided to the bendable portion 612. The elongated portion 611 and bendable portion 612 further have a cylindrical hollow portion 614, by which a surgical instrument 616 can be inserted from the proximal end of the elongated portion 611 to the distal end of the bendable portion 612. The continuum robot 610 also has a jacket 615 that covers the sides and tip of the bendable portion 612 and the hollow portion 614.

The proximal end of the jacket 615 is fixed to the distal end of the elongated portion 611, so when bending driving of the bendable portion 612 is performed, the outer side of the jacket 615 stretches, and the inner side of the jacket 615 is compressed, as illustrated in FIG. 26B. The surgical instrument 616 is also bent following the bendable portion 612. Accordingly, the bending rigidity of the bendable portion 612 is greater as compared to the continuum robots in the above-described first through fifth embodiments.

In the present embodiment, the following Assumptions 13 and 14 are set forth, and an elastic wire driving model is derived that takes into consideration the bending rigidity of the jacket 615 and surgical instrument 616. Assumption 13 is that the magnitude of bending moment of the jacket 615 and surgical instrument 616 is proportionate to the bending angle of the bendable portion 612. Assumption 14 is that the rotation axis of bending moment of the jacket 615 and surgical instrument 616 is parallel to the Y₂ axis.

Based on Assumption 13, the balance of moment on the Y₂ axis at the distal end of the bendable portion 612 can be expressed by the following Expression (63) in which items representing the bending moment of the jacket 615 and surgical instrument 616 have been added to Expression (54), in which k_{mc} represents the bending rigidity of the jacket 615 and kₘₜ represents the bending rigidity of the surgical instrument 616.

$$\begin{matrix}[ Math \text{.23} ] & \\ & ( 63 )\end{matrix}$$

$$\frac{{R}_{g}}{2} \{ {f}_{1 a} sin ( - {ζ}_{1} - \frac{π}{2} ) + {f}_{1 b} sin ( - {ζ}_{1} + \frac{π}{6} ) + {f}_{1 c} sin ( - {ζ}_{1} + \frac{5 π}{6} ) \} - {M}_{1 a 1} - {M}_{1 b 1} - {M}_{1 c 1} + {k}_{mc} {θ}_{1} + {k}_{mt} {θ}_{1}$$

Based on Assumption 14, the balance of moment on the X₂ axis is not affected by the rigidity of the jacket 615 and surgical instrument 616, so Expression (55) is appropriate for the continuum robot 610 according to the present embodiment as well. The bending rigidity k_{mc} Of the jacket 615 and the bending rigidity kₘₜ of the surgical instrument 616 may be identified through experimentation, or may be analytically derived.

### (6-2. Control System)

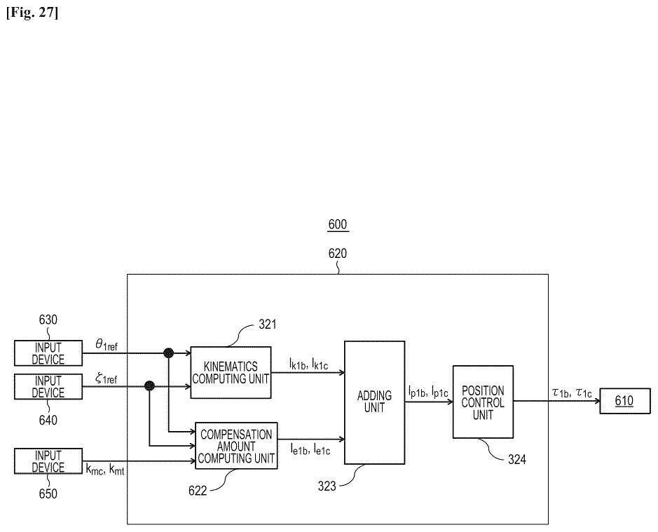

In the sixth embodiment, the displacement of the wires is derived taking into consideration the rigidity of the jacket 615 and surgical instrument 616, by performing iterative calculation the same as in the third embodiment, using Expression (63). FIG. 27 is a diagram illustrating an example of the functional configuration of a continuum robot control system 600 according to the sixth embodiment of the present invention. Configurations that are the same as configurations of the third embodiment illustrated in FIG. 16 are denoted by the same symbols, and detailed description thereof will be omitted.

The continuum robot control system 600 illustrated in FIG. 27 is configured including the continuum robot 610 illustrated in FIG. 26, a continuum robot control device 620, an input device 630, an input device 640, and an input device 650. The continuum robot control device 620 is configured including the kinematics computing unit 321, a compensation amount computing unit 622, the adding unit 323, and the position control unit 324.

The compensation amount computing unit 622 computes the compensation amounts l_{e1b} and l_{e1c} for each of the wires 1b and 1c, based on the target bending angle θ_{1ref} of the bendable portion 612 input from the input device 630, the target rotation angle ζ_{1ref} of the bendable portion 612 input from the input device 640, and the bending rigidity k_{mc} of the jacket 615 and the bending rigidity kₘₜ of the surgical instrument 616 input from the input device 650. Although the bending rigidity k_{mc} of the jacket 615 and the bending rigidity kₘₜ of the surgical instrument 616 is assumed to be constant in the present embodiment, an arrangement may be made where, when the rigidity has nonlinearity dependent on bending angle or rotation angle, rigidities corresponding to various bending angles and rotation angles are stored in a storage device before the procedure, and rigidities corresponding to target bending angles and target rotation angles are read out during the procedure. Alternatively, the operator may directly input the bending rigidity k_{mc} of the jacket 615 and the bending rigidity kₘₜ of the surgical instrument 616. Note that the kinematics computing unit 321, adding unit 323, and position control unit 324 are the same as in the third embodiment illustrated in FIG. 16, so description will be omitted.

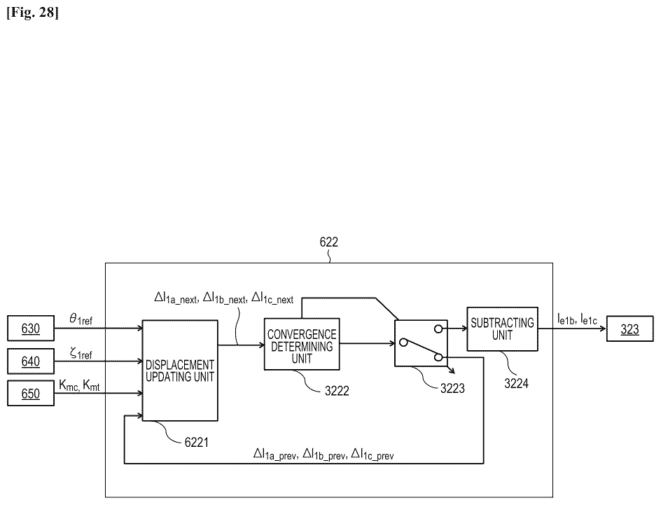

FIG. 28 is a diagram illustrating an example of the functional configuration of the compensation amount computing unit 622 illustrated in FIG. 27, according to the sixth embodiment of the present invention. Configurations in FIG. 28 that are the same as configurations illustrated in FIG. 27 and configurations of the third embodiment illustrated in FIG. 17 are denoted by the same symbols, and detailed description thereof will be omitted. The compensation amount computing unit 622 illustrated in FIG. 28 is configured including a displacement updating unit 6221, the convergence determining unit 3222, the output switching unit 3223, and the subtracting unit 3224.

In accordance with input of the target bending angle θ_{1ref} and target rotation angle ζ_{1ref}, the bending rigidity k_{mc} of the jacket 615 and bending rigidity kₘₜ of the surgical instrument 616, and first displacement candidate values Δl_{1a_prev}, Δl_{1b_prev}, and Δl_{1c_prev}, in displacement of the wires 1a, 1b, and 1c, the displacement updating unit 6221 updates to second displacement candidate values Δl_{1a_next}, Δl_{1b_next}, and Δl_{lc_next}. Note that the convergence determining unit 3222, output switching unit 3223, and subtracting unit 3224 are the same as in the third embodiment illustrated in FIG. 17, so description will be omitted.

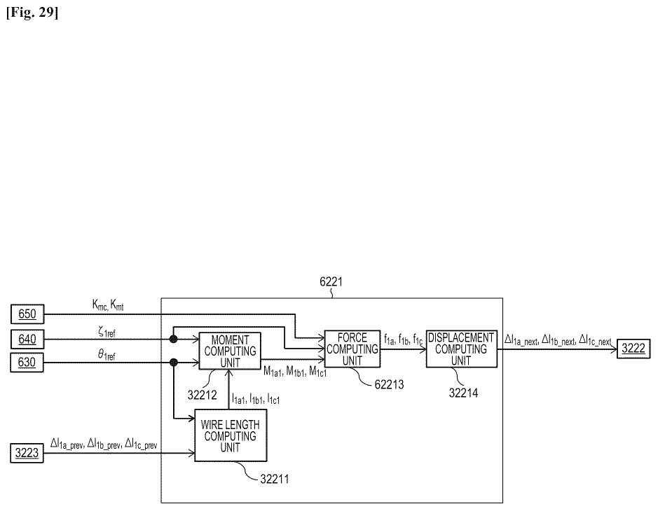

FIG. 29 is a diagram illustrating an example of the functional configuration of the displacement updating unit 6221 illustrated in FIG. 28, according to the sixth embodiment of the present invention. Configurations in FIG. 29 that are the same as configurations illustrated in FIG. 28 and configurations of the third embodiment illustrated in FIG. 18 are denoted by the same symbols, and detailed description thereof will be omitted. The displacement updating unit 6221 illustrated in FIG. 29 is configured including the wire length computing unit 32211, the moment computing unit 32212, a force computing unit 62213, and the displacement computing unit 32214.

The force computing unit 62213 calculates forces f₁ₐ, f_{1b}, and f_{1c} representing the tensile force on the wires 1a, 1b, and 1c, in accordance with the input of the bending rigidity km of the jacket 615 and bending rigidity kₘₜ of the surgical instrument 616, in addition to the input of the bending moments M₁ₐ₁, M_{1b1}, and M_{1c1}, of the multiple wires 1a, 1b, and 1c, using Expression (53) that expresses balance of force, and Expressions (55) and (63) that express balance of moment. Note that the wire length computing unit 32211, moment computing unit 32212, and the displacement computing unit 32214 are the same as in the third embodiment illustrated in FIG. 18, so description will be omitted.

Although a model is derived that includes the bending rigidity k_{mc} of the jacket 615 and bending rigidity kₘₜ of the surgical instrument 616 as shown in Expression (63) in the present embodiment, an arrangement may be made where displacement is calculated using an approximation expression where the bending rigidity kₘ of the wires in Expressions (10) and (57) is replaced with a rigidity kₘₑ that takes into consideration with the rigidity of the jacket 615 and surgical instrument 616 as well. Alternatively, the rigidity kₘₑ may be identified through experimentation, or a value obtained by dividing the bending rigidity kₘ of the jacket 615 and bending rigidity kₘₜ of the surgical instrument 616 by the wire count n may be added to the wire bending rigidity kₘ, as shown in the following Expression (64).

$$\begin{matrix}[ Math \text{.24} ] & \\ \text{?} = {k}_{m} + \frac{\text{?} + {k}_{mt}}{\text{?}} & ( 64 )\end{matrix}$$

$$\text{?} \text{indicates text missing or illegible when filed}$$

While the continuum robot control device 620 is applied to the continuum robot 610 having one bendable portion 612 that is three-dimensionally bendable in the present embodiment, application may be made to the continuum robot 110 that curves on a plane as in the first embodiment, or the continuum robot 410 that has multiple bendable portions 312 as in the fourth embodiment.

### (6-3. Simulation)

The control system according to the sixth embodiment illustrated in FIGS. 27 through 29 is applied to the continuum robot 610 that has the jacket 615, and through which the surgical instrument 616 has been inserted. The bending rigidity k_{mc} of the jacket 615 and bending rigidity kₘₜ of the surgical instrument 616 here are both assumed to be 0.33 Nm/rad.

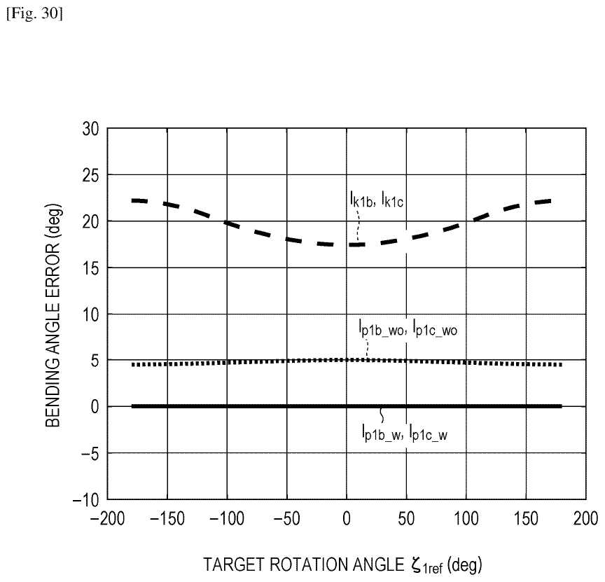

FIG. 30 is a characteristic diagram illustrating the relation between target rotation angle and bending angle error according to the sixth embodiment of the present invention. The horizontal axis in the characteristics diagram of FIG. 30 is the target rotation angle ζ_{1ref}, and the vertical axis is the bending angle error, in the same way as in FIG. 20.

Specifically, the dashed line shown at the top in FIG. 30 illustrates a comparative example of a case where the driving amounts l_{k1b} and k_{k1c} calculated using the kinematics model are used as control input. It can be understood that in this case, great angle error of the bendable portion 612 occurs dependent on the target rotation angle ζ_{1ref}. Also, the dotted line shown at the middle illustrates a comparative example of a case where the drive control amounts l_{p1b_wo} and 1_{p1c_wo} calculated using the continuum robot control device 320 according to the third embodiment are used as control input. It can be understood that in this case, angle error cannot be completely compensated for by the continuum robot control device 320 according to the third embodiment, since increase in rigidity of the bendable portion 612 due to the jacket 615 and surgical instrument 616 has not been taken into consideration. On the other hand, the solid line shown to the bottom represents response when using drive control amounts l_{p1b_w} and l_{p1c_w} computed using the continuum robot control device 620 according to the present embodiment, and it can be understood that the angle error of the bendable portion 612 is constantly 0, regardless of the target rotation angle ζ_{1ref}. From the above description, it can be understood that attitude error of the continuum robot 610 can be reduced by applying the control system according to the sixth embodiment illustrated in FIGS. 27 through 29, to the continuum robot 610 that has the jacket 615, and through which the surgical instrument 616 has been inserted.

### Seventh Embodiment

Next, a seventh embodiment of the present invention will be described. Description of items that are the same as in the above-described first through sixth embodiments will be omitted from the following description of the seventh embodiment, and description will be made regarding items that are different from the above-described first through sixth embodiments.

The continuum robot 310 according to the third embodiment illustrated in FIG. 14 has the actuators 311b and 311c connected to, out of the three wires 1a, 1b, and 1c, the wires 1b and 1c respectively, and the bendable portion 312 can be three-dimensionally bent. Accordingly, in a case where one of the two actuators 311b and 311c stops operating due to malfunction, the bendable portion 312 becomes capable of bending only on a plane, so the risk of tissue and the continuum robot 310 coming into contact increases. On the other hand, if all wires 1a, 1b, and 1c are connected to actuators, three-dimensional bending operations can be realized by a control system like that of the third embodiment even if one actuator happens to malfunction, and accordingly the continuum robot control system can be imparted redundancy. Accordingly, an example is illustrated in the seventh embodiment regarding a control system that is applicable to a continuum robot capable of driving all three wires 1a, 1b, and 1c.

### (7-1. Modelling)

The continuum robot used in the present embodiment differs from the continuum robot 310 according to the third embodiment, with regard to the point that the wire 1a illustrated in FIG. 14 is not connected to the fixing portion 311a, but rather to an actuator 311a′. The continuum robot where the wire 1a illustrated in FIG. 14 is connected to the actuator 311a′ instead of the fixing portion 311a will be described as a “continuum robot 710” used in the seventh embodiment.

In the continuum robot 710, using the actuators 311a′, 311b, and 311c to drive the wire 1a, wire 1b, and wire 1c respectively, by the same distance in the same direction, the length of the center axis of the bendable portion corresponding to the bendable portion 312 increases or decreases with the bending angle and rotation angle remaining constant. Accordingly, the length of the center axis length l_{1dc} of the bendable portion can be controlled with the continuum robot 710 according to the present embodiment, in addition to the bending angle θ₁ and the rotation angle ζ₁.

The configuration of the bendable portion of the continuum robot 710 used in the present embodiment has the same configuration as the bendable portion 312 used in the third embodiment, so a kinematics model and elastic wire model is derived for the continuum robot 710 using FIG. 15. When the bending angle of the bendable portion of the continuum robot 710 is θ₁ and the rotation angle is ζ₁, a distance Rₒₐ between the origin O₁ on the W₁-Z₁ plane and the wire 1b can be expressed by the following Expression (65).

$$\begin{matrix}[ Math \text{.25} ] & \\ {R}_{oa} = \frac{{R}_{g}}{2} cos ( {ζ}_{1} ) & ( 65 )\end{matrix}$$

Accordingly, the following Expression (66) holds between the center axis length l_{1dc} and the length l₁ₐ₁.

$$\begin{matrix}[ Math \text{.26} ] & \\ {l}_{1 a 1} = - \frac{{R}_{g} {θ}_{1}}{2} cos ( {ζ}_{1} ) + {l}_{1 dc} & ( 66 )\end{matrix}$$

Also, from Expressions (45) and (46), the length l_{1b1} and the length l_{1c1} can be expressed by Expressions (67) and (68) using length l_{1dc}.

$$\begin{matrix}[ Math \text{.27} ] & \\ {l}_{1 b 1} = \frac{{R}_{g} {θ}_{1}}{2} cos ( {ζ}_{1} - \frac{2 π}{3} ) + {l}_{1 d c} & ( 67 )\end{matrix}$$

$$\begin{matrix}[ Math \text{.28} ] & \\ {l}_{1 c 1} = \frac{{R}_{g} {θ}_{1}}{2} cos ( {ζ}_{1} - \frac{4 π}{3} ) + {l}_{1 d c} & ( 68 )\end{matrix}$$

Assuming that the wires 1a, 1b, and 1c are rigid in the longitudinal direction, the respective wire lengths l₁ₐ₁, l_{1b1}, and l_{1c1} are expressed as being the sums of the driving amounts lₖ₁ₐ, l_{k1b}, and l_{k1c}, derived from the kinematic model, and the length l₁₀. Accordingly, the driving amounts lₖ₁ₐ, l_{k1b}, and l_{k1c} are respectively expressed by the following Expressions (69), (70), and (71)

$$\begin{matrix}[ Math \text{.29} ] & \\ {l}_{k 1 a} = - \frac{{R}_{g} {θ}_{1}}{2} cos ( {ζ}_{1} ) + {l}_{1 d c} - {l}_{0} & ( 69 )\end{matrix}$$

$$\begin{matrix}[ Math \text{.30} ] & \\ {l}_{k 1 a} = - \frac{{R}_{g} {θ}_{1}}{2} cos ( {ζ}_{1} - \frac{2 π}{3} ) + {l}_{1 d c} - {l}_{0} & ( 70 )\end{matrix}$$

$$\begin{matrix}[ Math \text{.31} ] & \\ {l}_{k 1 c} = - \frac{{R}_{g} {θ}_{1}}{2} cos ( {ζ}_{1} - \frac{4 π}{3} ) + {l}_{1 d c} - {l}_{0} & ( 71 )\end{matrix}$$

Next, assuming that the wires 1a, 1b, and 1c stretch and contract in the longitudinal direction, the respective displacements Δl₁ₐ, Δl_{1b}, and Δl_{1c} are obtained. Based on Expressions (9) and (56), defining matrix Kₑ and vector f, Δl₁ respectively as shown in the following Expressions (72) and (73), Δl₁ is expressed as in the following Expression (74).

$$\begin{matrix}[ Math \text{.32} ] & \\ {K}_{e} = [ \begin{matrix}{k}_{e} & 0 & 0 \\ 0 & {k}_{e} & 0 \\ 0 & 0 & {k}_{e}\end{matrix} ] & ( 72 )\end{matrix}$$

$$f = {[ \begin{matrix}{f}_{1 a} & {f}_{1 b} & {f}_{1 c}\end{matrix} ]}_{T} ,$$

$$Δ {l}_{1} = {[ \begin{matrix}Δ {l}_{1 a} & Δ {l}_{} & {}_{1 b} & Δ {l}_{} & {}_{1 c}\end{matrix} ]}_{T} \qquad ( 73 )$$

$$f + {K}_{3} Δ {l}_{1} = 0 \qquad ( 74 )$$

Based on Expressions (10), (53) through (55) and (57), defining matrix A and vector mi respectively as shown in the following Expressions (75) and (76), the following Expression (77) holds.

$$\begin{matrix}[ Math \text{.33} ] & \\ A = [ \begin{matrix}1 & 1 & 1 \\ \frac{{R}_{g}}{2} sin ( - {ζ}_{1} - \frac{π}{2} ) & \frac{{R}_{g}}{2} sin ( - {ζ}_{1} - \frac{π}{6} ) & \frac{{R}_{g}}{2} sin ( - {ζ}_{1} + \frac{5 π}{6} ) \\ \frac{{R}_{g}}{2} sin ( - {ζ}_{1} ) & \frac{{R}_{g}}{2} sin ( - {ζ}_{1} + \frac{2 π}{3} ) & \frac{{R}_{g}}{2} sin ( - {ζ}_{1} + \frac{4 π}{3} )\end{matrix} ] & ( 75 )\end{matrix}$$

$$\begin{matrix}[ Math \text{.34} ] & \\ & ( 76 )\end{matrix}$$

$${m}_{1} = {[ 0 - {k}_{m} {θ}_{1} \{ \frac{1}{- \frac{{R}_{g} {θ}_{1}}{2} cos ( {ζ}_{1} ) + \text{?}} + \frac{1}{- \frac{{R}_{g} {θ}_{1}}{2} cos ( {ζ}_{1} - \frac{2 π}{3} ) + {l}_{1 dc}} + \frac{1}{- \frac{{R}_{g} {θ}_{1}}{2} cos ( {ζ}_{1} - \frac{4 π}{3} ) + {l}_{1 dc}} \} 0 ]}^{T}$$

$$Af + {m}_{1} = 0 \qquad ( 77 )$$

$$\text{?} \text{indicates text missing or illegible when filed}$$

### (7-2. Control System)

In the seventh embodiment, the drive control amounts lₚ₁ₐ, l_{p1b}, and l_{p1c} of the wires 1a, 1b, and 1c are computed so as to compensate for the stretching and contraction of the wires 1a, 1b, and 1c connected to the actuators 311a′, 311b, and 311c, respectively.

The drive control amounts lₚ₁ₐ, l_{p1b}, and l_{p1c} of the wires 1a, 1b, and 1c are found by the following Expressions (79) through (81) here.

$${l}_{p 1 a} = {l}_{k 1 a} - Δ {l}_{1 a} \qquad ( 79 )$$

$${l}_{p 1 b} = {l}_{k 1 b} - Δ {l}_{1 b} \qquad ( 80 )$$

$${l}_{p 1 c} = {l}_{k 1 c} - Δ {l}_{l c} \qquad ( 81 )$$

Accordingly, the compensation amounts lₑ₁ₐ, l_{e1b}, and l_{e1c} of the wires 1a, 1b, and 1c are respectively expressed by the following Expressions (82) through (84).

$${l}_{e 1 a} = - Δ {l}_{1 a} \qquad ( 82 )$$

$${l}_{e 1 b} = - Δ {l}_{1 b} \qquad ( 83 )$$

$${l}_{e 1 c} = - Δ {l}_{1 c} \qquad ( 84 )$$

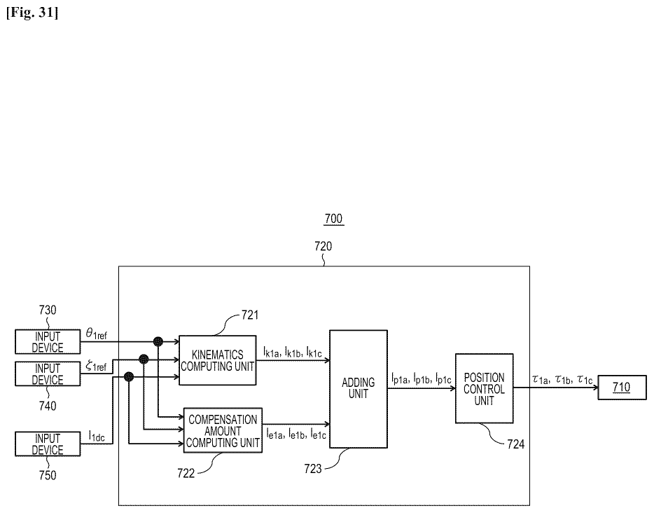

FIG. 31 is a diagram illustrating an example of the functional configuration of a continuum robot control system 700 according to the seventh embodiment of the present invention. The continuum robot control system 700 illustrated in FIG. 31 is configured including the continuum robot 710, a continuum robot control device 720, an input device 730, an input device 740, and an input device 750.

The continuum robot control device 720 sets the drive control amount lₚ₁ₐ for driving control of the wire 1a, the drive control amount l_{p1b} for driving control of the wire 1b, and the drive control amount l_{p1c} for driving control of the wire 1c, based on the target bending angle θ_{1ref} of the bendable portion of the continuum robot 710 input from the input device 730, the target rotation angle ζ_{1ref} of the bendable portion of the continuum robot 710 input from the input device 740, and the center axis length l_{1dc} input from the input device 750. The continuum robot control device 720 then outputs a driving command τ₁ₐ based on the drive control amount lₚ₁ₐ, a driving command τ_{1b} based on the drive control amount l_{p1b}, and a driving command τ_{1c} based on the drive control amount l_{p1c}, to the continuum robot 710 (or more specifically, the actuators 311a′, 311b, and 311c).

The continuum robot control device 720 illustrated in FIG. 31 is configured including a kinematics computing unit 721, a compensation amount computing unit 722, an adding unit 723, and a position control unit 724.

Based on the target bending angle θ_{1ref} of the bendable portion of the continuum robot 710 input from the input device 730, the target rotation angle ζ_{1ref} of the bendable portion of the continuum robot 710 input from the input device 740, and the center axis length l_{1dc} input from the input device 750, the kinematics computing unit 721 computes the driving amounts lₖ₁ₐ, l_{k1b}, and l_{k1c} based on the kinematic model, using Expressions (69) through (71).

The compensation amount computing unit 722 computes the displacements Δl₁ₐ, Δl_{1b}, and Δl_{1c} for each of the wires 1a, 1b, and 1c, using Expression (78), and further computes the compensation amounts lₑ₁ₐ, l_{e1b}, and l_{e1c}, using Expressions (82) through (84). The wire displacement Δl₁ is uniquely determined from the bending angle θ₁, rotation angle ζ₁, and center axis length l_{1dc}, which can be clearly understood from the right side of Expression (78). Accordingly, the compensation amount computing unit 722 according to the present embodiment can compute the displacements Δl₁ₐ, Δl_{1b}, and Δl_{1c} without using iterative calculation, in the same way as with the first embodiment.

The adding unit 723 adds the driving amounts lₖ₁ₐ, l_{k1b}, and l_{k1c} calculated by the kinematics computing unit 721 and the compensation amounts lₑ₁ₐ, l_{e1b}, and l_{e1c} calculated by the compensation amount computing unit 722, thereby computing the driving control amounts lₚ₁ₐ, l_{p1b}, and l_{p1c}, for each of the wires 1a, 1b, and 1c.

The position control unit 724 outputs a driving command τ₁ₐ based on the drive control amount lₚ₁ₐ, a driving command th based on the drive control amount l_{p1b}, and a driving command ti based on the drive control amount l_{p1c}, to the continuum robot 710 (or more specifically, the actuators 311a′, 311b, and 311c).

While the present embodiment uses the center axis length l_{1dc} as the length of the bendable portion to be input from the input unit 750 to the kinematics computing unit 721 and compensation amount computing unit 722, one of the wire lengths l₁ₐ₁, l_{1b1}, and l_{1c1} may be used for example. Also, although the continuum robot control device 720 is applied to the continuum robot 710 having one bendable portion that can be bent threedimensionally in the present embodiment, application may be made to the continuum robot 110 that curves on a plane as in the first embodiment, or the continuum robot 410 that has multiple bendable portions 312 as in the fourth embodiment.

### (7-3. Simulation)

The control system according to the seventh embodiment is applied to the continuum robot 710 that can drive all three wires 1a, 1b, and 1c. Note that both the length l_{1dc} and the length l₁₀ are 10 mm in the present embodiment.

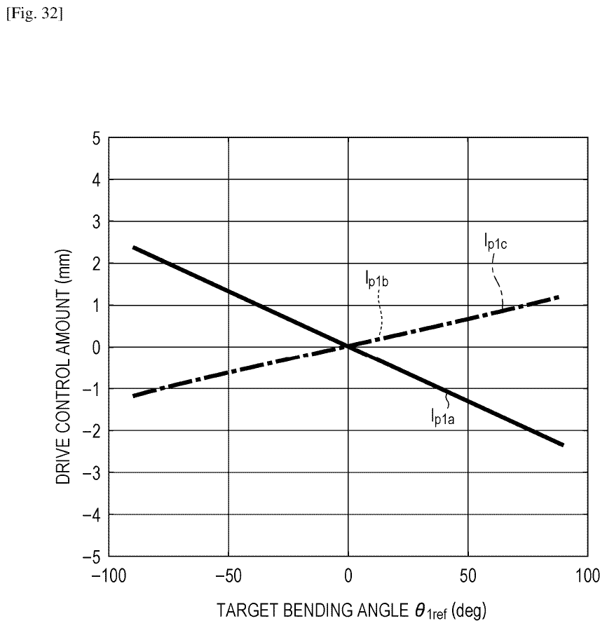

FIG. 32 is a characteristics diagram illustrating the relation between target bending angle and driving control amounts lₚ₁ₐ, l_{p1b}, and 1_{p1c} according to the seventh embodiment of the present invention. The horizontal axis in the characteristics diagram of FIG. 32 is the target bending angle θ_{1ref}, and the vertical axis is the wire driving distance.

Specifically, FIG. 32 shows the driving control amounts lₚ₁ₐ, l_{p1b}, and l_{p1c}, by a solid line, dotted line, and dashed line, respectively, in a case where the target rotation angle ζ_{1ref} is 0 degrees, and the target bending angle θ_{1ref} is changed from −90 degrees to 90 degrees. The continuum robot control device 720 according to the seventh embodiment drives the three wires 1a, 1b, and 1c, so when the target bending angle changes, the driving control amounts of all of the wires 1a, 1b, and 1c, including the wire 1a, increase or decrease, as illustrated in FIG. 32. Also, the continuum robot control device 720 according to the present embodiment computes the driving control amounts lₚ₁ₐ, l_{p1b}, and l_{p1c} so that the center axis length is constantly l_{1dc}, so in a case where the target bending angle θ_{1ref} is positive, the driving direction of the wire 1a situated on the inner side as to the center axis is in the pulling direction of the wire (negative direction), and the driving direction of the wires 1b and 1c situated on the outer side as to the center axis is in the pushing direction of the wire (positive direction).

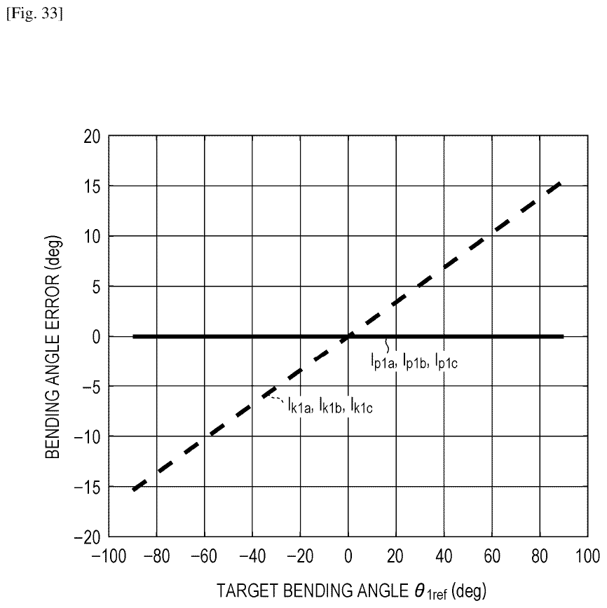

FIG. 33 is a characteristics diagram illustrating the relation between target rotation angle and bending angle error according to the seventh embodiment of the present invention. The horizontal axis in the characteristics diagram of FIG. 33 is the target bending angle θ_{1ref}, and the vertical axis is the bending angle error.

Specifically, FIG. 33 shows bending angle error in a case where the target rotation angle ζ_{1ref} is 0 degrees and the target bending angle θ_{1ref} is changed from-90 degrees to 90 degrees. The dashed line in FIG. 33 illustrates a comparative example of a case where the driving amounts lₖ₁ₐ, l_{k1b}, and l_{k1c} calculated using the kinematics model are used as control input. It can be understood that in this case, a great angle error occurs. On the other hand, the solid line illustrates angle error in a case of using the drive control amounts lₚ₁ₐ, l_{p1b}, and l_{p1c} calculated using the continuum robot control device 720 according to the seventh embodiment as control input, and it can be seen that the angle error of the bendable portion of the continuum robot 710 is constantly 0. From the above, it can be understood that attitude error of the continuum robot 710 that can drive all three wires 1a, 1b, and 1c can be reduced, by applying the control system according to the seventh embodiment illustrated in FIG. 31.

### Other Embodiments

The above-described first through sixth embodiments have been arrangements where the wire 1a of each bendable portion is fixed, and the driving control amount of the other wire 1b or the like being set, thereby compensating for the displacement of the wire 1a that is fixed. That is to say, the above-described first through sixth embodiments are arrangements of continuum robot control devices that control operations of a continuum robot having a bendable portion that can be bent by driving part of multiple wires. However, the present invention may also be applied to an arrangement of a continuum robot control device that controls operations of a continuum robot having a bendable portion that can be bent by driving all of multiple wires, as in the seventh embodiment. In the case of this form, application can be made to arrangements where all wires are driven to compensate for displacement of the wires themselves, and arrangements where there are wires that compensate driving amount (predetermined wire) and wires that do not compensate driving amount (other wire).

Although friction between wires and wire guides, and between wires and the elongated portion have not been taken into consideration in the above-described first through seventh embodiments, the displacement may be computed taking frictional force into consideration, if the frictional force is known. Specifically, an arrangement may be made where the displacement is computed using the following Expression (85) where frictional forces fᵣ₁ₐ and f_{r1b} are added to the left side of Expression (9), instead of Expression (9).

$${f}_{1 a} + {f}_{r 1 a} = - {k}_{e} Δ {l}_{1 a} , \qquad ( 85 )$$

$${f}_{1 b} + {f}_{r 1 b} = - {k}_{e} Δ {l}_{1 b} ,$$

Note that in this embodiment as well, the driving amount of the wires can be compensated for using compensation amounts calculated based on displacement of any one of multiple wires pertaining to the same bendable portion, in the same way as with the first embodiment.

The present invention can be realized by processing of supplying a program that realizes one or more functions of the above-described embodiments to a system or device via a network or storage medium, and one or more processors of a computer of the system or device reading out and executing the program. The present invention can also be realized by a circuit that realizes one or more functions (e.g., an application specific integrated circuit (ASIC)). The program, and a computer-readable storage medium storing the program, are encompassed by the present invention.

The above-described embodiments of the present invention are only exemplary illustrations of carrying out the present invention, and the technical scope of the present invention should not be restrictively interpreted by these. That is to say, the present invention can be carried out in various forms without departing from the technical spirit or primary features thereof.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A continuum robot control device configured to control operations of a continuum robot having a bendable portion that is bent by driving at least part of a plurality of wires, the continuum robot control device comprising:
a first computing device configured to compute a driving amount of the at least part of the plurality of wires, based on a target bending angle that is a target value for a bending angle of the bendable portion, and based on a target rotation angle that is a target value for a rotation angle of the bendable portion;
a second computing device configured to compute a compensation amount for compensation of an error in the driving amount due to stretching and contraction of the wires, based on the target bending angle, the target rotation angle and a displacement of one of the plurality of wires at the target bending angle and the target rotation angle; and
a setting device configured to set a driving control amount of performing driving control of the at least part of the plurality of wires, based on the driving amount calculated at the first computing device and the compensation amount calculated at the second computing device.

2. The continuum robot control device according to claim 1, wherein the continuum robot has a plurality of the bendable portions disposed serially to each other,
wherein the first computing device computes the driving amount based on the target bending angle, for each bendable portion of the plurality of bendable portions,
wherein the second computing device computes the compensation amount for each bendable portion of the plurality of bendable portions, and
wherein the setting device sets the driving control amount based on the driving amount calculated by the first computing device and the compensation amount calculated by the second computing device, for each bendable portion of the plurality of bendable portions.

3. The continuum robot control device according to claim 1 wherein the second computing device calculates displacement of one wire of the plurality of wires in accordance with input of the target bending angle, and wherein the second computing device computes a compensation amount of another one of the wires based on the calculated displacement of the one wire of the plurality of wires.

4. The continuum robot control device according to claim 1, wherein the second computing device is further configured to:
compute wire length for each of the plurality of wires at the bendable portion, in accordance with input of the target bending angle and the first candidate value,
compute bending moment for each of the plurality of wires, in accordance with input of the target bending angle and the wire length of the plurality of wires,
compute, in accordance with input of the bending moment at the plurality of wires, tensile force at the plurality of wires, and
compute the second candidate value in accordance with input of the tensile force.

5. The continuum robot control device according to claim 1, wherein the second computing device the updating unit is further configured to:
update a candidate value in displacement of the plurality of wires including the another wire, as the first candidate value,
compute wire length for each of the plurality of wires at the bendable portion, in accordance with input of the target bending angle and the first candidate value,
compute bending moment for each of the plurality of wires, in accordance with input of the target bending angle, a target rotation angle that is a target value of a rotation angle of the bendable portion, and the wire length of the plurality of wires,
compute tensile force of the plurality of wires, in accordance with input of the bending moment at the plurality of wires, and
compute the second candidate value in the displacement of the plurality of wires including the another wire, in accordance with input of the tensile force at the plurality of wires.

6. The continuum robot control device according to claim 1, wherein the length of the wires is ten times or more the length of the bendable portion.

7. A continuum robot control method configured to control operations of a continuum robot having a bendable portion that is bent by driving at least part of a plurality of wires, the continuum robot control method comprising:
computing a driving amount of the at least part of the plurality of wires, based on a target bending angle that is a target value for a bending angle of the bendable portion and based on a target rotation angle that is a target value for a rotation angle of the bendable portion;
computing a compensation amount for compensation of an error in the driving amount due to stretching and contraction of the wires, based on the target bending angle, the target rotation angle and a displacement of one wire of the plurality of wires at the target bending angle and the target rotation angle; and
setting a driving control amount of performing driving control of the at least part of the plurality of wires, based on the calculated driving amount and the calculated compensation amount.

8. A computer-readable storage medium storing a program configured to cause a computer to function as the devices of the continuum robot control device according to claim 1.

9. The continuum robot control device according to claim 1, wherein the compensation amount for the stretching and contraction of first wire of the plurality of wires is based on amount of displacement of the first wire and a second wire of the plurality of wires.
